(19) United States
(12) Patent Application Publication
(10) Pub. No.: US 2023/0351990 A1
(43) Pub. Date: Nov. 2, 2023

(54) SYSTEMS AND METHODS FOR TRANSPOSING SPOKEN OR TEXTUAL INPUT TO MUSIC

(71) Applicants: YAO THE BARD, LLC., Plymouth, MA (US); NEW YORK UNIVERSITY, New York, NY (US)

(72) Inventors: Leonardus H.T. Van Der Ploeg, Newton, MA (US); Halley Young, Philadelphia, PA (US); Joshua Armenta, Mexico City (MX); Bhubaneswar Mishra, Great Neck, NY (US)

(21) Appl. No.: 17/918,696

(22) PCT Filed: Apr. 13, 2021
(86) PCT No.: PCT/US2021/027107

§ 371 Date: Oct. 13, 2022

Related U.S. Application Data
(60) Provisional application No. 63/009,166, filed on Apr. 13, 2020.

(51) Int. Cl. G10H 1/00; A61B 5/16

(52) U.S. Cl. CPC G10H 1/0025; A61B 5/165; G10H 2210/111; G10H 2240/085; G10H 2250/455; G10H 2250/311

(57) ABSTRACT

Described herein are real-time musical translation devices (RETMs) and methods of use thereof. Exemplary uses of RETMs include optimizing the understanding and/or recall of an input message for a user and improving a cognitive process in a user.

### CROSS-REFERENCE TO RELATED APPLICATIONS

This application claims priority under 35 U.S.C. § 119(e) to U.S. Provisional Application Ser. No. 63/009,166, titled “Systems And Methods For Transposing Spoken Or Textual Input To Music,” filed on Apr. 13, 2020, which is hereby incorporated by reference in its entirety.

### BACKGROUND

The present disclosure is directed to systems and methods for transposing spoken or textual input to music. For millennia, humans have used music, and in particular vocal songs and melodies, to convey information in a manner that heightens interest and facilitates comprehension and long-term recall of the information conveyed. The timing and variations in pitch and rhythm in a song may signal to the listener what information is important and how different concepts in the text are related to each other, causing the listener to retain and understand more of the information than if it was merely spoken. The unique ability of song to convey information that is distinctly processed by the brain from non-musical spoken words is supported by brain imaging results which have shown that different patterns of brain activity occur for spoken words when compared to words in song. The findings highlighting unique cognitive processing of words in song, are supported by applications where, in addition to their entertainment value, songs may be taught to children to assist with learning and remembering the number of days in a month, the states and their capitals, or other pieces of information that may otherwise elude understanding or memory retention.

Separately, but relatedly, persons with a cognitive impairment, behavioral impairment, or learning impairment may find it easier to comprehend and recall information when conveyed as a song or melody. For example, a passage of text read in a normal speaking tone by the student or an instructor may not be comprehended or recalled, whereas the same passage of text when sung may be more easily comprehended and recalled by individuals, including persons having impairments including, for example, dyslexia, aphasia, autism spectrum disorder, Alzheimer's disease, dementia, Down's syndrome, Prader Willi syndrome, Smith Magenis syndrome, indications that include learning disability and/or intellectual disability, Parkinson's disease, anxiety, stress, schizophrenia, brain surgery, surgery, stroke, trauma, or other neurological disorder. Exposure to information “coded” in music is anticipated to lead, over the long term, to enhanced verbal IQ, quantitative measures of language comprehension, and quantitative measures of the ability to interact with care providers.

While users with selected clinical impairments may benefit from information being sung, the general population of instructors, care providers, teachers and the like may not have the capability or willingness to sing the information to be conveyed. Even if instructors do have such willingness and skills, transforming text or voice to a musical score takes time and effort if word recognition and comprehension are to be optimally retained. Furthermore, for the case of text, the instructor's physical presence could be required for the text to be heard. For example, an instructor may be required to be present to read a text and sing the information to be conveyed. In addition, different individuals and/or different disorders may respond to different styles and natures of music (i.e., genre, tempo, rhythm, intervals, key, chord structure, song structure), meaning that even for a given passage of information, a one-size-fits-all approach may be inadequate. While it is possible to compose, pre-record and play back information being sung, such an arrangement is inflexible in that it does not allow for the music or the information being conveyed to be adjusted in real time, or near real time, such as in response to student questions or needs.

### SUMMARY

A device and/or software are provided for receiving real-time or near-real-time input (e.g., a textual, audio, or spoken message) containing information to be conveyed, and converting that input to a patterned musical message, such as a melody, intended to facilitate a learning or cognitive process of a user. The musical message may be performed in real-time or near-real time. In the examples described here, the application and device are described as a dedicated Real Time Musical Translation Device (RETM), wherein “device” should be understood to refer to a system that incorporates hardware and software components, such as mobile applications. It will be appreciated, however, that the application may also be performed on other audio-input and -output capable devices, including a mobile device such as a smart phone, tablet, laptop computer, and the like, that has been specially programmed.

The RETM may allow the user to have some control and/or selection regarding the musical themes that are preferred or that can be chosen. For example, a user may be presented with a list of musical genres, moods, styles, or tempos, and allowed to filter the list of songs according to the user's selection, which will be taken to transfer routine spoken words or text to the musical theme, in real or near-real time. In another example, the user may identify one or more disorders that the patterned musical message is intended to be adapted for, and the RETM may select a genre and/or song optimized for that disorder. In yet another example, a user may be “prescribed,” by a medical care provider, a genre for instance as part of a musical therapy, suitable for treating the user's disorder. It will be appreciated that as used herein, “genre” is intended to encompass different musical styles and traditions originating from different time periods, locations, or cultural groups, as well as systematic differences between artists within a given time period. Genres may include, for example, rock, pop, R&B, hip-hop, rap, country, nursery rhymes, or traditional music such as Gregorian chants or Jewish Psalm tones, as well as melodies fitting a particular class of tempo (“slow”, “medium”, or “fast”), mood (“cheerful”, “sad”, etc.), or predominant scale (“major” or “minor”) or other quantifiable musical property. User preferences, requirements, and diagnoses may be learned and stored by the device, such that an appropriate song or genre may be suggested and/or selected by the RETM in an intuitive and helpful manner. In some embodiments, machine learning and/or artificial intelligence algorithms may be applied to enable the RETM to learn, predict, and/or adapt to user preferences, requirements, and diagnoses including collecting and applying user-data that describe a user's physiological condition including heart rate, eye movements, breathing, muscle-tone, movement, pharmacodynamic markers of RETM efficacy.

In some embodiments, the selections regarding genre and/or disorder may be used to match portions of a timed text input to appropriate melody segments in order to generate a patterned musical message.

It will be appreciated that while the patterned musical message generated and output by the RETM is referred to here as a “melody” for the sake of simplicity, the patterned musical message is not necessarily a melody as defined in musical theory, but may be any component of a piece of music that when presented in a given musical context is musically satisfying and/or that facilitates word or syntax comprehension or memory, including rhythm, harmony, counterpoint, descant, chant, particular spoken cadence (e.g., beat poetry), or the like, as exemplified in rhythmic training, phonemic sound training or general music training for children with dyslexia. It will also be appreciated that the musical pattern may comprise an entire song, one or more passages of the song, or simply a few measures of music, such as the refrain or “hook” of a song. More generally, music may be thought of in this context as the melodic transformation of real-time spoken language or text to known and new musical themes by ordering tones and sounds in succession, in combination, and in temporal relationships to produce a composition having unity and continuity. For instance, in cases of stroke causing lesion to the left-hemisphere, particularly near language-related areas such as Broca's area, any patterning that leads to a more musical output, including all musical or prosodic components above, may lead to increased ability to rely on intact right-hemisphere function to relearn the ability to speak and attain comprehension. In the case of dyslexia, any one of these added musical dimensions to the text may provide alternative pathways for comprehension.

According to some embodiments, recognition and/or comprehension of the words presented in song can be over 95%, or over 99%, or over 99.5%, or over 99.9% using the methods and/or devices described herein. It will be appreciated that any significant improvement in comprehension can lead to significant improvements of quality of life in cases such as post-stroke aphasia, where patients will need to communicate with their caretakers and other individuals, in dyslexia, where individuals may struggle less in educational settings, or for any indication or non-clinical setting where quality of life is hindered by the inability to efficiently communicate or attain information through spoken or textual sources.

While scenarios involving an “instructor” and a “student” are described here for clarity purposes, it should be understood that the term “user” of the device, as referred to herein, encompasses any individual that may use the device, such as a regular reader or person communicating, an instructor, a teacher, a physician, a nurse, a therapist, a student, a parent or guardian of said student, or a care provider. A user of the device may or may also be referred to herein as a “subject.” A user may be a child or an adult, and may be either male or female. In an embodiment, the user is a child, e.g., an individual 18 years of age or younger. In an embodiment, the user may have an indication, such as a learning disability, Alzheimer's disease, or may be recovering from a stroke. In an embodiment, the RETM may be used to facilitate general understanding and comprehension of routine conversation.

It is also to be appreciated that real-time translation of information to patterned musical messages may benefit typically developing/developed users as well as those with a disorder or other condition. Furthermore, the real-time or near real-time translation of spoken or textual language to music made possible by these systems and methods provide advantages beyond the therapeutic uses discussed here. For example, the RETM may be used for musical or other entertainment purposes, including music instruction or games.

In one aspect, the present disclosure features a method of transforming textual input to a musical score comprising (i) receiving a timed text input; (ii) receiving a melody; (iii) generating a plurality of transformed melodies from the melody; (iv) determining, for each of the plurality of transformed melodies, a fit metric between the respective transformed melody and the timed text input; (v) selecting the transformed melody from the plurality of transformed melodies based on the fit metric of the selected transformed melody; and (vi) generating a patterned musical message from the selected transformed melody and the timed text input. In an embodiment, the method further comprises splitting the melody into a plurality of melody subsequences (e.g., determined using heuristics for segmentation). In an embodiment, the method further comprises generating the plurality of transformed melodies from the plurality of melody subsequences. In an embodiment, the method further comprises splitting the timed text input into a plurality of timed text subsequences. In an embodiment, the method further comprises determining, for each of the timed text sequences, a fit metric between the respective timed text sequence and each of the plurality of transformed melodies. In an embodiment, splitting the timed text input into the plurality of timed text subsequences is iteratively performed using dynamic programming.

In some embodiments, determining, for each of the plurality of transformed melodies, the fit metric for the respective transformed melody and the timed text input comprises one or more of (i) determining an onset pattern of the transformed melody; (ii) determining a set of optimal syllable lengths of the timed text input; (iii) mapping the onset pattern of the transformed melody to a first real-valued vector; (iv) mapping the optimal syllable lengths of the timed text input to a second real-valued vector; and (v) determining a cosine similarity between the first real-valued vector and the second real-valued vector. In an embodiment, determining comprises two of (i)-(v). In an embodiment, determining comprises three of (i)-(v). In an embodiment, determining comprises four of (i)-(v). In an embodiment, determining comprises each of (i)-(v).

In some embodiments, the first real-valued vector and the second real-valued vector are derived from sparse vectors, in which the only non-zero entries are at the onsets of the melody or timed text input. In some embodiments, dense vectors are derived from the sparse vectors using a neural autoencoder, and wherein a loss function of the neural autoencoder is an edit distance loss function.

In some embodiments, the timed text input is received as text input by a user of a device. In some embodiments, the timed text input is derived from speech spoken by a user and received at a microphone of a device. In some embodiments, the timed text input is derived from text that is derived from speech spoken by a user and received at a microphone of a device.

In some embodiments, generating the plurality of transformed melodies from the melody comprises augmenting, diminishing, inverting, elaborating, transposing, or simplifying at least one portion of the melody. In some embodiments, receiving the melody comprises automatically composing a plurality of melody subsequences using at least one of constraint programming, logic programming, or generative neural models.

In another aspect, the present disclosure features a system for transforming textual input to a musical score comprising a processor; and a memory configured to store instructions that when executed by the processor cause the processor to (i) receive a timed text input; (ii) receive a melody; (iii) generate a plurality of transformed melodies from the melody; (iv) determine, for each of the plurality of transformed melodies, a fit metric between the respective transformed melody and the timed text input; (v) select the transformed melody from the plurality of transformed melodies based on the fit metric of the selected transformed melody; and (vi) generate a patterned musical message from the selected transformed melody and the timed text input.

In another aspect, the present disclosure features a non-transitory computer-readable medium storing sequences of instruction for transforming textual input to a musical score, the sequences of instruction including computer executable instructions that instruct at least one processor to (i) receive a timed text input; (ii) receive a melody; (iii) generate a plurality of transformed melodies from the melody; (iv) determine, for each of the plurality of transformed melodies, a fit metric between the respective transformed melody and the timed text input; (v) select the transformed melody from the plurality of transformed melodies based on the fit metric of the selected transformed melody; and (vi) generate a patterned musical message from the selected transformed melody and the timed text input.

According to aspects of the disclosure, a method of transforming textual input to a musical score is provided comprising receiving a timed text input, receiving a melody, generating a plurality of transformed melodies from the melody, determining, for each of the plurality of transformed melodies, a fit metric between the respective transformed melody and the timed text input, selecting the transformed melody from the plurality of transformed melodies based on the fit metric of the selected transformed melody, and generating a patterned musical message from the selected transformed melody and the timed text input.

In various examples, generating the plurality of transformed melodies from the melody comprises splitting the melody into a plurality of melody subsequences (e.g., determined using heuristics for segmentation), and generating the plurality of transformed melodies from the plurality of melody subsequences. In some examples, the method includes splitting the timed text input into a plurality of timed text subsequences, and determining, for each of the timed text sequences, a fit metric between the respective timed text sequence and each of the plurality of transformed melodies. In at least one example, splitting the timed text input into the plurality of timed text subsequences is iteratively performed using dynamic programming.

In various examples, generating the plurality of transformed melodies from the melody is performed using a stochastic model. In some examples, the stochastic model is a Markov model. In at least one example, determining, for each of the plurality of transformed melodies, the fit metric for the respective transformed melody and the timed text input comprises determining an onset pattern of the transformed melody, determining a set of optimal syllable lengths of the timed text input, mapping the onset pattern of the transformed melody to a first real-valued vector, mapping the optimal syllable lengths of the timed text input to a second real-valued vector, and determining a cosine similarity between the first real-valued vector and the second real-valued vector.

In various examples, the first real-valued vector and the second real-valued vector are derived from sparse vectors, in which the only non-zero entries are at the onsets of the melody or timed text input. In some examples, dense vectors are derived from the sparse vectors using a neural autoencoder, and wherein a loss function of the neural autoencoder is an edit distance loss function. In at least one example, the timed text input is received as text input by a user of a device. In various examples, the timed text input is derived from speech spoken by a user and received at a microphone of a device.

In some examples, the timed text input is derived from text that is derived from speech spoken by a user and received at a microphone of a device. In at least one example, generating the plurality of transformed melodies from the melody comprises augmenting, diminishing, inverting, elaborating, transposing, or simplifying at least one portion of the melody. In various examples, receiving the melody comprises automatically composing a plurality of melody subsequences using at least one of constraint programming, logic programming, or generative neural models.

According to aspects of the disclosure, a system for transforming textual input to a musical score is provided comprising a processor, and a memory configured to store instructions that when executed by the processor cause the processor to receive a timed text input, receive a melody, generate a plurality of transformed melodies from the melody, determine, for each of the plurality of transformed melodies, a fit metric between the respective transformed melody and the timed text input, select the transformed melody from the plurality of transformed melodies based on the fit metric of the selected transformed melody, and generate a patterned musical message from the selected transformed melody and the timed text input.

According to aspects of the disclosure, a non-transitory computer-readable medium storing sequences of instruction for transforming textual input to a musical score, the sequences of instruction including computer executable instructions that instruct at least one processor to receive a timed text input, receive a melody, generate a plurality of transformed melodies from the melody, determine, for each of the plurality of transformed melodies, a fit metric between the respective transformed melody and the timed text input, select the transformed melody from the plurality of transformed melodies based on the fit metric of the selected transformed melody, and generate a patterned musical message from the selected transformed melody and the timed text input.

According to aspects of the disclosure, a method of modulating a state of a user is provided comprising receiving, via a user interface, a first selection of a text, selecting, based on at least one parameter of the user, a mental exercise, converting, based on the selected mental exercise, the first selection of the text to at least one of a first sung sequence or a first chanted sequence, and outputting the at least one of the sung sequence or the chanted sequence via a transducer.

In some examples, the at least one parameter of the user includes one of a gender of the user, a body-mass index of the user, a time of day, a genetic polymorphism of the user, a cultural background of the user, or a language chosen by the user. In various examples, the method includes receiving feedback information indicative of the state of the user, modifying, based on the at least one parameter of the user and the feedback information, the mental exercise to optimize the mental exercise for modulation of the state of the user, receiving, via the user interface, a second selection of a text, converting, based on the modified mental exercise, the second selection of the text to at least one of a second sung sequence or a second chanted sequence, and outputting the at least one of the second sung sequence or the second chanted sequence via the transducer.

In at least one example, the feedback information is biological or physiological feedback information. In various examples, modifying the mental exercise includes executing a Bayesian-optimization technique based on the biological or physiological feedback information. In some examples, executing the Bayesian-optimization technique includes optimizing the mental exercise to modulate the state of the user. In at least one example, the state of the user includes a mental mood of the user. In various examples, modulating the mental mood of the user includes modulating a predicted altered state of at least one region of the brain involved in managing stress, involved in verbalization information indicative of the state of the user, or involved in depression of the user including the bed nucleus of the stria terminalis (BNST) of the user.

In some examples, outputting the at least one of the second sung sequence or the second chanted sequence via the transducer is predicted to modulate the state of the BNST of the user. In at least one example, the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers. In various examples, the feedback includes user-input information.

In some examples, the user-input information includes a user selection of a metric indicative of a satisfaction of the user with the mental exercise. In at least one example, the user-input information includes metadata indicative of one or more inputs provided by the user. In various examples, the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.

According to aspects of the disclosure, a non-transitory computer-readable medium storing thereon sequences of computer-executable instructions for modulating a state of a user is provided, the sequences of computer-executable instructions including instructions that instruct at least one processor to receive, via a user interface, a first selection of a text, select, based on at least one parameter of the user, a mental exercise, convert, based on the selected mental exercise, the first selection of the text to at least one of a first sung sequence or a first chanted sequence, and output the at least one of the sung sequence or the chanted sequence via a transducer.

In various examples, the at least one parameter of the user includes one of a gender of the user, a body-mass index of the user, a time of day, a genetic polymorphism of the user, a cultural background of the user, or a language chosen by the user. In at least one example, the instructions further instruct the at least one processor to receive feedback information indicative of the state of the user, modify, based on the at least one parameter of the user and the feedback information, the mental exercise to optimize the mental exercise for modulation of the state of the user, receive, via the user interface, a second selection of a text, convert, based on the modified mental exercise, the second selection of the text to at least one of a second sung sequence or a second chanted sequence, and output the at least one of the second sung sequence or the second chanted sequence via the transducer.

In some examples, the feedback information is biological or physiological feedback information. In various examples, modifying the mental exercise includes executing a Bayesian-optimization technique based on the biological or physiological feedback information. In at least one example, executing the Bayesian-optimization technique includes optimizing the mental exercise to modulate the state of the user. In some examples, the state of the user includes a mental mood of the user. In various examples, modulating the mental mood of the user includes modulating a predicted altered state of at least one region of the brain involved in managing stress, involved in verbalization information indicative of the state of the user, or involved in anxiety or depression of the user including the bed nucleus of the stria terminalis (BNST) of the user. In at least one example, outputting the at least one of the second sung sequence or the second chanted sequence via the transducer is predicted to modulate the state of the BNST of the user.

In some examples, the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers. In at least one example, the feedback includes user-input information. In various examples, the user-input information includes a user selection of a metric indicative of a satisfaction of the user with the mental exercise. In some examples, the user-input information includes metadata indicative of one or more inputs provided by the user. In at least one example, the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.

The details of one or more embodiments of the invention are set forth herein. Other features, objects, and advantages of the invention will be apparent from the Detailed Description, the Figures, the Examples, and the Claims.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples and are incorporated in and constitute a part of this specification, but are not intended as a definition of the limits of a particular example. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. In the figures:

FIG. 1 is a functional block diagram of a real-time musical translation device (RETM) according to one embodiment;

FIG. 2A depicts a process for operating the application and/or a device according to one embodiment;

FIG. 2B depicts a process for operating the application and/or a device according to one embodiment;

FIG. 3 depicts an exemplary user interface according to one embodiment;

FIG. 4 depicts a process for operating the application and/or a device according to one embodiment;

FIG. 5 depicts an exemplary user interface according to one embodiment;

FIG. 6 shows an example computer system with which various aspects of the present disclosure may be practiced;

FIG. 7 shows an example storage system capable of implementing various aspects of the present disclosure;

FIG. 8 depicts a process for operating the application and/or a device according to one embodiment; and

FIG. 9 illustrates a process 900 of modulating a mental state of an individual using audible outputs according to an example

### DETAILED DESCRIPTION

### Real-Time Musical Translation Device

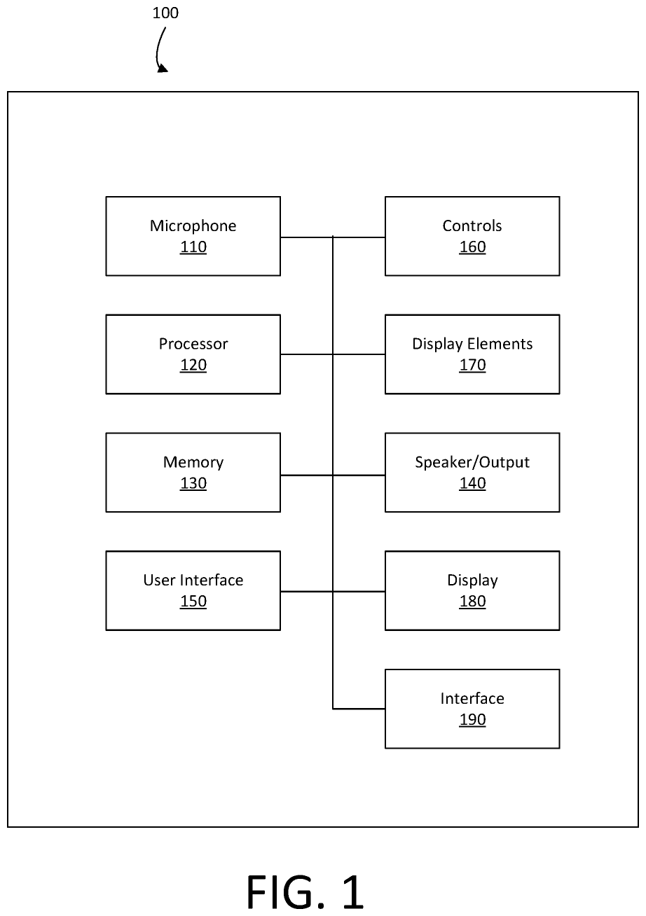

A block diagram of an exemplary real-time musical translation device (RETM) 100 is shown in FIG. 1. The RETM 100 may include a microphone 110 for receiving an audio input (e.g., spoken information) from a user, and may also be configured to receive voice commands for operating the RETM 100 from the user via the microphone. A processor 120 and a memory 130 are in communication with each other and the microphone to receive, process through selected algorithms and code, and/or store the audio input or information or signals derived therefrom, and ultimately to generate the patterned musical message. A user interface 150, along with controls 160 and display elements 170, allow a user to interact with the RETM 100 (e.g., by picking a song to use as a basis for generating the patterned musical message). A speaker or other output 140 may act as a transducer (i.e., convert the patterned musical message to an audio signal) or may provide the patterned musical message device to another device (e.g., headphones or an external speaker). Optionally, a display device 180 may display visual and/or textual information designed to reinforce and/or complement the patterned musical message. An interface 190 allows the RETM 100 to communicate with other devices, including through local connection (e.g., Bluetooth) or through a LAN or WAN (e.g., the Internet).

The microphone 110 may be integrated into the RETM 100, or may be an external and/or separately connectable microphone, and may have any suitable design or response characteristics. For example, the microphone 110 may be a large diaphragm condenser microphone, a small diaphragm condenser microphone, a dynamic microphone, a bass microphone, a ribbon microphone, a multi-pattern microphone, a USB microphone, or a boundary microphone. In some examples, more than one microphone may be deployed in an array. In some embodiments, the microphone 110 may not be provided (or if present may not be used), with audio input received from an audio line in (e.g., AUX input), or via a wired or wireless connection (e.g., Bluetooth) to another device.

The processor 120 and/or other components may include functionality or hardware for enhancing and processing audio signals, including, for example, signal amplification, analog-to-digital conversion/digital audio sampling, echo cancellation, audio mastering, or other audio processing, etc., which may be applied to input from the microphone 110 and/or output to the speaker 140 of the RETM 100. As discussed in more detail below, the RETM 100 may employ pitch- and time-shifting on the audio input, with reference to a score and/or one or more rules, in order to convert a spoken message into the patterned musical message.

The memory 130 is non-volatile and non-transitory and may store executable code for an operating system that, when executed by the processor 120, provides an application layer (or user space), libraries (also referred to herein as “application programming interfaces” or “APIs”) and a kernel. The memory 130 also stores executable code for various applications, including the processes and sub-processes described here. Other applications may include, but are not limited to, a web browser, email client, calendar application, etc. The memory may also store various text files and audio files, such as, but not limited to, text to be converted to a patterned musical message; a score or other notation, or rules, for the patterned musical message; raw or processed audio captured from the microphone 110; the patterned musical message itself; and user profiles or preferences. Melodies may be selected and culled according to their suitability for optimal text acceptance. This selection may be made by a human (e.g., the user or an instructor) and/or automatically by the RETM or other computing device, such as by using a heuristic algorithm.

The source or original score may be modified to become optimally aligned with voice and/or text, leading to the generated score, which, includes the vocal line, is presented by the synthesized voice and presents the text as lyrics. The generated score, i.e. the musical output of the RETM, may include pitch and duration information for each note and rest in the score, as well as information about the structure of the composition represented by the generated score, including any repeated passages, key and time signature, and timestamps of important motives. The generated score may also include information regarding other parts of the composition not included in the patterned musical message. The score may include backing-track information, or may provide a link to a prerecorded backing track and/or accompaniment. For example, the RETM 100 may perform a backing track along with the patterned musical message, such as by simulating drums, piano, backing vocals, or other aspects of the composition or its performance. In some embodiments, the backing track may be one or more short segments that can be looped for the duration of the patterned musical message. In some examples, the score is stored and presented according to a technical standard for describing event messages, such as the Musical Instrument Digital Interface (MIDI) standard. Data in the score may specify the instructions for music, including a note's notation, pitch, velocity, vibrato, and timing/tempo information.

A user interface 150 may allow the user to interact with the RETM 100. For example, the user (e.g., instructor or student) may use user interface 150 to select a song or genre used in generating the patterned musical message, to display text that the user may read to provide the audio input, to receive feedback from a user (for example, indicating a user's satisfaction with a patterned musical message generated by the RETM 100), to display an animated visualization or other graphic intended to enhance the user's enjoyment or comprehension, and so forth. Other controls 160 may also be provided, such as physical or virtual buttons, capacitive sensors, switches, or the like, for controlling the state and function of the RETM 100. Similarly, display elements 170 may include LED lights or other indicators suitable for indicating information about the state or function of the RETM 100, including, for example, whether the RETM 100 is powered on, whether it is currently receiving audio input or playing back the patterned musical message. Such information may also be conveyed by the user interface 150. Tones or other audible signals may also be generated by the RETM 100 to indicate such state changes.

The user interface 150 allows one or more users to select a musical pattern and/or ruleset as discussed herein. In some examples, different users may have different abilities to control the operation of the RETM 100 using the user interface 150. For example, whereas a first user (e.g., an instructor) may be allowed to select a disorder, a genre, and/or a song, a second user (e.g., a student) may be constrained to choosing a particular song within a genre and/or set of songs classified for a particular disorder by the first user or otherwise. In some examples, the disorder is a neurological disorder. In other examples, the disorder is a non-neurological disorder (e.g., a physiological disorder). In other examples the user simply aims to increase the ability to learn or comprehend. In this manner, a first user can exercise musical preferences within a subset of musical selections useful for treating a second user. In an embodiment, a first user can exercise musical preferences within a subset of musical selection useful for treating a plurality of users, such as a second user, a third user, or a fourth user.

In some examples, the user may interact with the RETM 100 using other interfaces in addition to, or in place of, user interface 150. For example, the RETM 100 may allow for voice control of the device (“use ‘rock & roll’”), and may employ one or more wake-words allowing the user to indicate that the RETM 100 should prepare to receive such a voice command.

The display 180 may also be provided, either separately or as part of the user interface 150, for displaying visual or textual information that reinforces and/or complements the information content of the text or voice or spoken words of the patterned musical message. In some embodiments, the display 180 may be presented on an immersive device such as a virtual reality (VR) or augmented reality (AR) headset.

The interface 190 allows the RETM 100 to communicate with other devices and systems. In some embodiments, the RETM 100 has a pre-stored set of data (e.g., scores and backing tracks); in other embodiments, the RETM 100 communicates with other devices or systems in real time to process audio and/or generate the patterned musical message. In various examples, the RETM 100 may receive feedback information corresponding to a user via one or more devices or systems via the interface 190. For example, feedback information indicative of a biological state of a user (for example, a breathing pattern of a user, a heart rate of a user, brain-scan information of a user, verbal feedback from a user, haptic feedback from a user, and so forth) may be acquired by one or more devices and/or systems and sent to the RETM 100 via the interface 190. As discussed in greater detail below, feedback information may be used to refine the manner by which the RETM 100 generates patterned musical messages.

Communications can be achieved via one or more networks, such as, but are not limited to, one or more of WiMax, a Local Area Network (LAN), Wireless Local Area Network (WLAN), a Personal area network (PAN), a Campus area network (CAN), a Metropolitan area network (MAN), a Wide area network (WAN), a Wireless wide area network (WWAN), enabled with technologies such as, by way of example, Global System for Mobile Communications (GSM), Personal Communications Service (PCS), Digital Advanced Mobile Phone Service (D-Amps), Bluetooth, Wi-Fi, Fixed Wireless Data, 2G, 2.5G, 3G, 4G, IMT-Advanced, pre-4G, 3G LTE, 3GPP LTE, LTE Advanced, mobile WiMax, WiMax 2, WirelessMAN-Advanced networks, enhanced data rates for GSM evolution (EDGE), General packet radio service (GPRS), enhanced GPRS, iBurst, UMTS, HSPDA, HSUPA, HSPA, UMTS-TDD, 1×RTT, EV-DO, messaging protocols such as, TCP/IP, SMS, MMS, extensible messaging and presence protocol (XMPP), real time messaging protocol (RTMP), instant messaging and presence protocol (IMPP), instant messaging, USSD, IRC, or any other wireless data networks or messaging protocols.

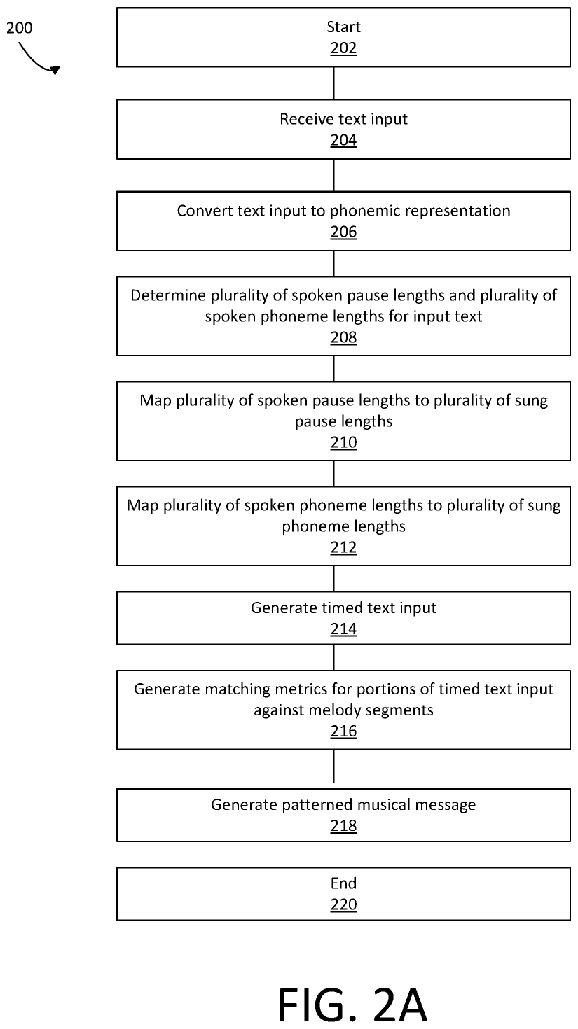

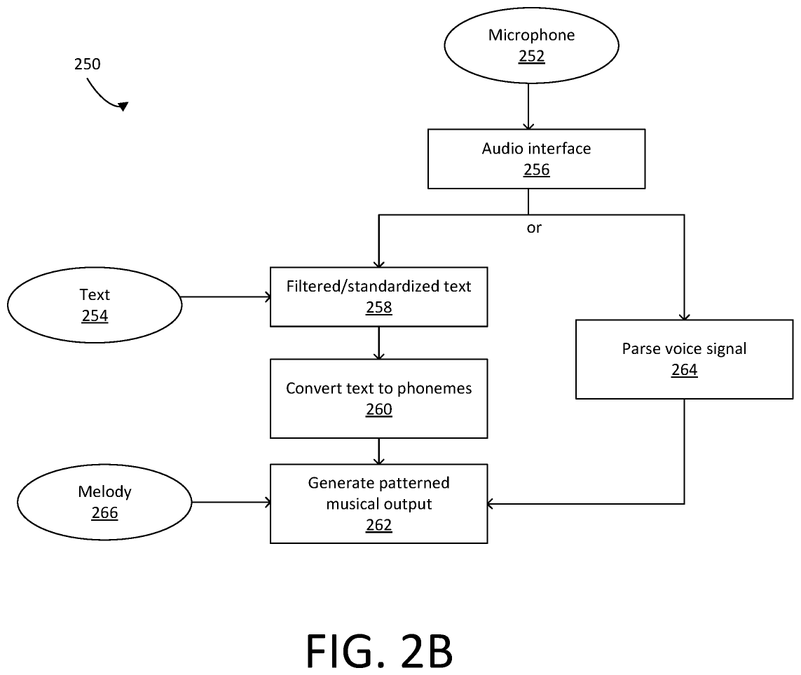

A method 200 of transposing spoken or textual input to a patterned musical message is shown in FIG. 2A. For purposes of this disclosure, it will be appreciated that “transpose” should be understood to encompass modifications to pitch as well as timing/tempo information, velocity, vibrato, timbre, and any other characteristics of musical notes or phrases.

At step 202, the method begins.

At step 204, text input is received. Text input may be received, for example, by accessing a text file or other computer file such as an image or photo, in which the text is stored. The text may be formatted or unformatted. The text may be received via a wired or wireless connection over a network, or may be provided on a memory disk. In other embodiments, the text may be typed or copy-and-pasted directly into a device by a user. In still other embodiments, the text may be obtained by capturing an image of text and performing optical character recognition (OCR) on the image. The text may be arranged into sentences, paragraphs, and/or larger subunits of a larger work. In various examples, a received text input or a portion thereof (for example, a phrase from a larger text) is selected by a user at act 204.

At step 206, the text input is converted into a phonemic representation, as can be represented by any standard format such as ARPABET, IPA or SAMPA. This may be accomplished, in whole or in part, using free or open source software, such as Phonemizer, and/or the Festival Speech Synthesis System developed and maintained by the Centre for Speech Technology Research at the University of Edinburgh. However, in addition certain phonemes in certain conditions (e.g., surrounded by other phonemes) are to be modified so as to be better comprehended as song. The phonemic content may be deduced by a lookup table mapping (spoken phoneme, spoken phoneme surroundings) to (sung phoneme). In some cases the entire preceding or consequent phoneme is taken into account when determining a given phoneme, while in other cases only the onset or end of the phoneme is considered.

In some examples, a series of filters may be applied to the text input to standardize or optimize the text input. For example, filters may be applied to convert abbreviations, currency signs, and other standard shorthand to text more suited for conversion to speech.

At step 208, a plurality of spoken pause lengths and a plurality of spoken phoneme lengths are determined for the text input. The length of the pauses and the phonemes represented in the text input may be determined with the help of open source software or other sources of information regarding the prosodic, syntactic, and semantic features of the text or voice. The process may involve a lookup table that synthesizes duration information about phonemes and pauses between syllables, words, sentences, and other units from other sources which describe normal speech. In some examples, the spoken length of phonemes may be determined and/or categorized according to their position in a larger syntactic unit (e.g., a word or sentence), their part of speech, or their meaning. In some examples, a dictionary-like reference may provide a phoneme length for specific phonemes and degrees of accent. For example, some phonemes may be categorized as having a phoneme length of less than 0.1 seconds, less than 0.2 seconds, less than 0.3 seconds, less than 0.4 seconds, or less than 1.0 seconds. Similarly, some pauses may be categorized according to their length during natural spoken speech, based upon their position within the text or a subunit thereof, the nature of phonemes and/or punctuation nearby in the text; or other factors.

At step 210, the plurality of spoken pause lengths is mapped to a respective plurality of sung pause lengths. Spoken pause lengths within a given duration range may be mapped to a level (e.g., “Level 1”, “Level 2”, etc.) associated with a corresponding level of sung pause lengths. For example, a Level 1 spoken pause (as discussed above) in spoken text may be mapped to a Level 1 sung pause, which may have a longer or shorter duration than the corresponding spoken pause. In some examples, any Level 1 spoken pause may be mapped to an acceptable range of Level 1 sung pauses. For example, a Level 1 spoken pause may be mapped to a range of Level 1 sung pauses of between 0.015 to 0.08 seconds or between 0.03 to 0.06 seconds. Similarly, a Level 2 spoken pause may be mapped to a sung pause of between 0.02 to 0.12 seconds or between 0.035 to 0.1 seconds. A Level 3 spoken pause may be mapped to a sung pause of between 0.05 to 0.5 seconds or between 0.1 to 0.3 seconds; and a Level 4 spoken pause may be mapped to a sung pause of between 0.3 to 1.5 seconds or between 0.5 to 1.0 seconds. In various examples, act 210 may include mapping the plurality of spoken pause lengths to a respective plurality of chanted pause lengths in addition to, or in lieu of, mapping the plurality of spoken pause lengths to a respective plurality of sung pause lengths. Furthermore, in some examples, the plurality of sung pause lengths may include chanted pause lengths.

At step 212, the plurality of spoken phoneme lengths is mapped to a respective plurality of sung phoneme lengths. The mapping may represent, for a spoken phoneme of a given length, a range of optimal lengths for the phoneme when sung. In some examples, a lookup table may be used, such as the following:

| Spoken Phoneme Length | Optimal Sung Phoneme Length |
|---|---|
| <0.1 seconds | 0.1 to 0.5 seconds |
| <0.2 seconds | 0.3 to 0.7 seconds |
| <0.3 seconds | 0.35 to 0.8 seconds |
| >=0.3 seconds | 0.4 to 0.9 seconds |

In another example, a broader range of values may be used:

| Spoken Phoneme Length | Optimal Sung Phoneme Length |
|---|---|
| <0.1 seconds | 0.05 to 0.7 seconds |
| <0.2 seconds | 0.2 to 0.9 seconds |
| <0.3 seconds | 0.3 to 1.0 seconds |
| >=0.3 seconds | 0.35 to 1.5 seconds |

It will be appreciated that the plurality of spoken pause lengths and the plurality of spoken phoneme lengths applied in steps 210 and 212, respectively, may be determined with reference to one or more parameters. Those parameters may include optimal breaks between sentences, optimal tempo, optimal time signature, optimal pitch range, and optimal length of phonemes, where optimality is measured with respect to facilitating comprehension and/or recollection. In some cases, a number of these factors may be applied, possibly with relative weights, in mapping the plurality of spoken pause lengths and the plurality of spoken phoneme lengths. In various examples, act 212 may include mapping the plurality of spoken phoneme lengths to a respective plurality of chanted phoneme lengths in addition to, or in lieu of, mapping the plurality of spoken phoneme lengths to a respective plurality of sung phoneme lengths. Furthermore, in some examples, the plurality of sung phoneme lengths may include chanted phoneme lengths.

Certain constraints may be imposed on the plurality of spoken pause lengths and the plurality of spoken phoneme lengths. In particular, spoken pause lengths and spoken phoneme lengths determined in the previous steps may be adjusted according to certain constraints in order to optimize comprehension and musicality. The constraints may be set based on the frequency/commonality of the word, or on its position within a sentence or clause, such as a “stop” word. For example, a constraint may be enforced that all phonemes in stop words must have a length of <=0.6 seconds. A stop word, as used herein, may be natural language words which have very little meaning, such as “and”, “the”, “a”, “an”, and similar words. Similarly, a constraint may be enforced that all phonemes in words that do not appear in the list of the most frequent 10,000 words must have a length of >=0.2 seconds. In another example, a constraint may be enforced that a pause after a stop word that does not end a sentence cannot be greater than 0.3 seconds.

At step 214, a timed text input is generated from the plurality of sung pause lengths and the plurality of sung phoneme lengths. In particular, each phoneme and pause in the text input is stored in association with its respective optimal timing (i.e., length) information determined in the previous steps. The timed text input (i.e., the text input and associated timing information) may be stored in an array, a record, and/or a file in a suitable format. In one example, a given phoneme in the timed text input may be stored as a record along with the lower and upper optimal length values, such as the following:

{“dh-ax-s”, 0.1, 0.5}

where the phoneme “dh-ax-s” (an ARPABET representation of the pronunciation of the word “this”) has been assigned an optimal sung phoneme length of between 0.1 and 0.5 seconds.

At step 216, a plurality of matching metrics is generated for each of a respective plurality of portions of the timed text input against a plurality of melody segments. The plurality of melody segments may be accessed in a MIDI file or other format. In addition to a melody line, a musical score or other information for providing an accompaniment to the melody may be accessed. For example, a stored backing track may be accessed and prepared to be played out in synchronization with the melody segments as described in later steps.

In particular, the timed text input may be broken up into portions representing sentences, paragraphs of text, or other units. Each portion is then compared to a plurality of melody segments, with each melody segment being a musical line having its own pitch and timing information.

Each melody segment may be thought of as the definition of a song, melody, or portion thereof, and may comprise a score as discussed above. For example, the melody segment may include, for each note in the melody, a number of syllables associated with the note, a duration of the note, a pitch of the note, and any other timing information for the note (including any rests before or after the note). While reference is made to a “pitch” of the note, it will be appreciated that the pitch may not be an absolute pitch (i.e., 440 Hz), but rather may be a relative pitch as defined by its position within the entire melody. For example, the melody segment may indicate that a particular note within the melody should be shifted to note with integer pitch 69 (equivalent to the letter note “A” in the fourth octave), but if it is deemed impossible to pronounce an A in fourth octave, the entire melody may be shifted downwards, so that each subsequent note it lowered by the same amount.

Other methods of musical corrective action may also be undertaken to enhance comprehension of the generated audio output. For example, the pitch (and all subsequent pitches) may be shifted to the appropriate note as an audio input message (i.e., the user's speaking voice), or some number of pitches above or below that original note, with the goal of sounding as natural as possible. In some example, the RETM may attempt to shift the pitches of the song by a particular number of semitones based on the nature of the disorder, the original pitch of the speaker's voice, or based on some determination that performance in that octave will be aesthetically pleasing.

For each comparison of a portion of a timed text input to a melody segment, a matching metric is generated representing the “fit” of the portion of the timed text input to the corresponding melody segment. For example, a melody segment with notes whose timing aligns relatively closely with the timing information of the corresponding portion of the timed text input may be assigned a higher matching metric than a melody segment that does not align as well timing-wise. A melody segment having the highest matching metric for a portion of the timed text input may be selected for mapping onto by the portion of the timed text input in subsequent steps.

The melody segments may be selected based on their harmonic and rhythmic profiles, such as their tonic or dominant scale qualities over the course of the melody. A subset of available melody segments may be chosen as candidates for a particular timed text input based on similar or complimentary musical qualities to ensure melodic coherence and appeal. In some examples, a user (e.g., an instructor) may be permitted to select a tonal quality (e.g., major or minor key) and/or tempo using a graphical or voice interface.

In some embodiments, a dynamic programming algorithm may be employed to determine which phonemes or words within the timed text input are to be matched with which melody segments or notes thereof. The algorithm may take into account linguistic features as well as their integration with musical features. For example, the algorithm may apply the timed text input to a melody segment such that a point of repose in the music (e.g., a perfect authentic cadence, commonly written as a “PAC”) is reached where there is a significant syntactic break. As another example, the algorithm may prevent breaking up stop words such as “the” with their following constituents; may favor harmonic tension following the syntax of the text. As another example, the algorithm may favor a longer duration for words assumed to be more rare and/or harder to hear in order to optimize comprehension and musicality.

A score function may be used by the dynamic programming algorithm in some embodiments for purposes of generating the matching metric between the portion of the timed text input and melody segment. The score function may weigh individual criteria, and the weights may be automatically set, dynamically adjustable, or adjustable by a user. In one example, one criterion may be the difference between the sung phoneme length(s) and the constraints imposed by the corresponding melody segment. In some embodiments, this length criterion may account for 50% of the score function. The length criterion may take into account the fit of the melody segment to the sung phoneme length as determined in steps 214 and 216 (80%), as well as syntactic/stop word analysis (10%), and word rarity (10%).

Another criterion taken into account in the scoring metric may be the degree to which pauses occur between complete clauses (30%). This may be determined by using a phrase structure grammar parser to measure the minimum depth of a phrase structure parsing of the sentence at which two sequential elements in the same chunking at that level are divided by the melody. If the depth is greater than or equal to some constant determined by the phrase structure grammar parser used (e.g., 4 for the open-source benepar parser), such a placement of the pause may be penalized.

Another criterion taken into account in the scoring metric may be the existence of unresolved tension only where the clause is incomplete (20%). A melody segment may be penalized where it causes a sentence or independent clause to end on the dominant or leading tone, or on a note with a duration of <1 beat.

In some examples, where none of the melody segment fit the portion of the timed text or voice input to a suitable degree, the timed text or voice input may be split into two or more subportions and the process repeated in an effort to locate one or a series of melody segments that fits each subportion of timed text or voice input to an acceptable degree.

At step 218, a patterned musical message is generated from the timed text or voice input and the plurality of melody segments based at least in part on the plurality of matching metrics. For example, each phoneme of the timed text input may be pitch shifted according to the corresponding notes(s) in the melody segment. The phoneme is set to the melody using phonetic transcription codes, such as ARPABET. The patterned musical message, with or without accompaniment, may then be output as a sound file, such as a .WAV or .MP3 file suitable for output by a playback device. The patterned musical message may be encoded with timestamps indicating a relative or absolute time at which each portion (e.g., note) of the melody is to be output.

Continuing with step 218, after or concurrent with output of the patterned musical message, visual or textual information may optionally be presented to reinforce or complement the patterned musical message. For example, the RETM may cause to be displayed, on a display screen or on-head display (such as a virtual reality or augmented reality display-enabled headset), the wording or imaging reflective of wording currently being output as part of the patterned musical message. In some embodiments, text corresponding to the currently played phoneme or the larger unit in which it is contained (e.g., word or sentence) may be highlighted or otherwise visually emphasized in order to enhance comprehension or recall. Identification of the currently played phoneme may be performed with reference to a timestamp associated a respectively timestamp associated with each phoneme in the patterned musical message.

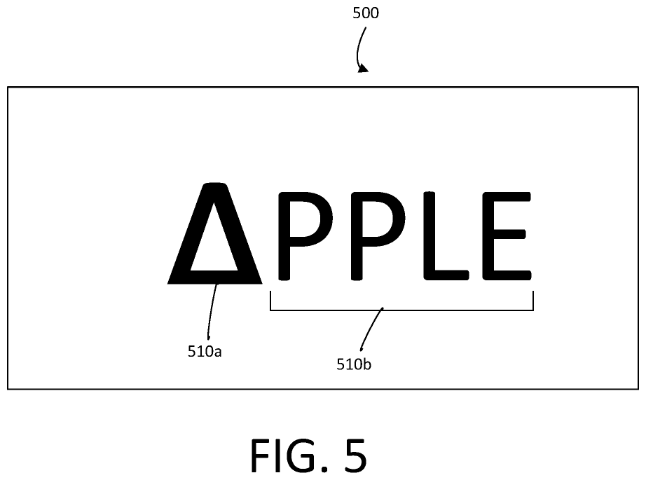

In some examples, characters in text being displayed may have their appearance modified in a way intended to optimize cognition and/or recall. An example screenshot 500 is shown in FIG. 5. In that example, the word “APPLE” is shown, but with the letter “A” (shown at 510a) being modified, having lowered and extended the horizontal feature of the letter. The remaining letters 510b are unchanged in appearance. Such and similar modified, and partial forms of any letters may be stored in association with one or more disorders, and displayed only when appropriate to treat such disorders. Other examples of modifications to characters include size, font face, movement, timing, or location relative to the other characters. In other examples, visual representations of the word (e.g., a picture of an apple when the word “apple” is sung in the patterned musical message) may be shown on the display. In some embodiments, virtual reality or augmented reality elements may be generated and displayed.

At step 220, the method ends.

According to some embodiments, the method 200 may be performed using a RETM (e.g., RETM 100 as seen in FIG. 1). The RETM may be a dedicated device, or may be the user's mobile device executing special-programmed software. In some examples, the user may be undergoing treatment with selected pharmacotherapeutics or behavioral treatments, or the user may be provided with or otherwise directed to use the RETM in combination with a drug or other therapeutic treatment intended to treat a disorder.

In some embodiments as described above, the input message may be textual input received from the user via a physical or virtual keyboard, or may be accessed in a text file or other file, or over a network. In other embodiments, the input text may be provided or derived from spoken or textual input by the user. In one example, the input message may be speech captured by a microphone (e.g., microphone 110) and stored in a memory (memory 130). In some examples, the intermediate step of parsing the input message spoken by the user into components parts of speech may be performed as a precursor to or in conjunction with step 206 as discussed above. In other examples, parsing the spoken input into text may be modified or omitted, and the waveform of the input message itself may simply be pitch-shifted according to certain rules and/or constraints as discussed below. In either case, it will be appreciated that a user's spoken input message may be mapped to and output as a melody in real-time or near-real-time as discussed herein.

An example block diagram for processing a variety of input messages is shown in FIG. 2B. For example, text input 254 may be received from a user and filtered and standardized at processing block 258, converted to phonemes at processing block 260, and used to generate a patterned musical message at processing block 262 based on a provided melody 266, according to the techniques described herein. In another example, spoken input is received at a microphone 252 and provided to an audio interface 256. Speech captured by the microphone 252 may undergo any number of pre-processing steps, including high pass, low pass, notch, band pass or parametric filtering, compression, expansion, clipping, limiting, gating, equalization, spatialization, de-essing, de-hissing, and de-crackling. In some embodiments, the audio input may be converted to text (e.g., for display on a device) using speech-to-text language processing techniques aimed at enhancing language comprehension.

The spoken input may then be converted to text using voice/speech recognition algorithms and processed in the same manner as the text 254 in processing blocks 258, 260, and 262.

In another embodiment, the spoken input may be directly parsed at processing block 264 without the intermediate step of converting to text. The audio input message may be parsed or processed in a number of ways at processing block 264. In some examples, waveform analysis allows the system to delineate individual syllables or other distinct sounds where they are separated by (even brief) silence as revealed in the waveform, which represents the audio input message as a function of amplitude over time. In these embodiments, syllables may be tagged by either storing them separately or by storing a time code at which they occur in the audio input message. Other techniques may be used to identify other parts of speech such as phonemes, words, consonants, or vowels, which may be detected through the use of language recognition software and dictionary lookups.

In some embodiments, the system may be configured to operate in a real-time mode; that is, audio input received at the microphone, or textual input received by the system, is processed and converted to a portion of the patterned musical message nearly instantaneously, or with a lag so minimal that it is either not noticeable at all or is slight enough so as not to be distracting. Input may be buffered, and the steps 202-220 may be performed repeatedly on any buffered input, to achieve real-time or near-real time processing. In these embodiments, the most recent syllable of the audio input message may continuously be detected and immediately converted to a portion of the patterned musical message. In other embodiments, the system may buffer two or more syllables to be processed. In some embodiments, the time between receiving the audio or text input message and outputting the patterned musical message should be vanishingly small so as to be virtually unnoticeable to the user. In some examples, the delay may be less than 2 seconds, and in further examples, the delay may be less than 0.5 seconds. In some examples, the delay may be less than 5 seconds, or less than 10 seconds. While the translation of spoken voice or text into song using the RETM may lengthen its presentation and thus lead to the termination of the song more than 10 seconds after the speaker finishes speaking in the case of a long utterance, the flow of song will be smooth and uninterrupted and will begin shortly after the speaker begins speaking.

In some embodiments, melody segments may be modified in order to more optimally fit the timed text input. The melody segments may be musically transformed using a stochastic model. In particular, several variants of a melody segment may be generated, and the results compared to the timed text input using, for example, geometric hashing. The variant representing the best fit for the timed text input (e.g., in terms of number of phonemes and timing) may be selected for use with the timed text input to generate the patterned musical message. The stochastic model may include a Markov model, a hidden Markov model, a Markov Decision Process (MDP) model, a Partially Observed Markov Decision Process (POMDP) model, a stochastic model, a SDE (Stochastic Diffusion equations) model, an auto-regressive model, a generalized auto regressive conditional heteroscedastic (GARCH) model, an econometric model, an automata model, a deterministic model, a probabilistic model, a non-deterministic automata model, a Kripke model, a Buchi automata model, a Rabin automata model, an ODE model, a hybrid automata model, a circuit model, a neural net model, a deep neural net (DNN) model, an RNN model, a LSTM NN model, a program synthesis model, a straight line program model, and a Turing-complete model.

In one example, the melody segments may be modified during the processes 200 and/or 250 as described above. For example, the melody segments may be modified after the timed text input is generated at step 214. Comparison of the variants to the timed text input may be performed, for example, concurrent with or as part of generating matching metrics at step 216. Generating the patterned musical melody may be performed, for example, concurrent with or as part of generating the patterned musical melody in step 216 of method 200.

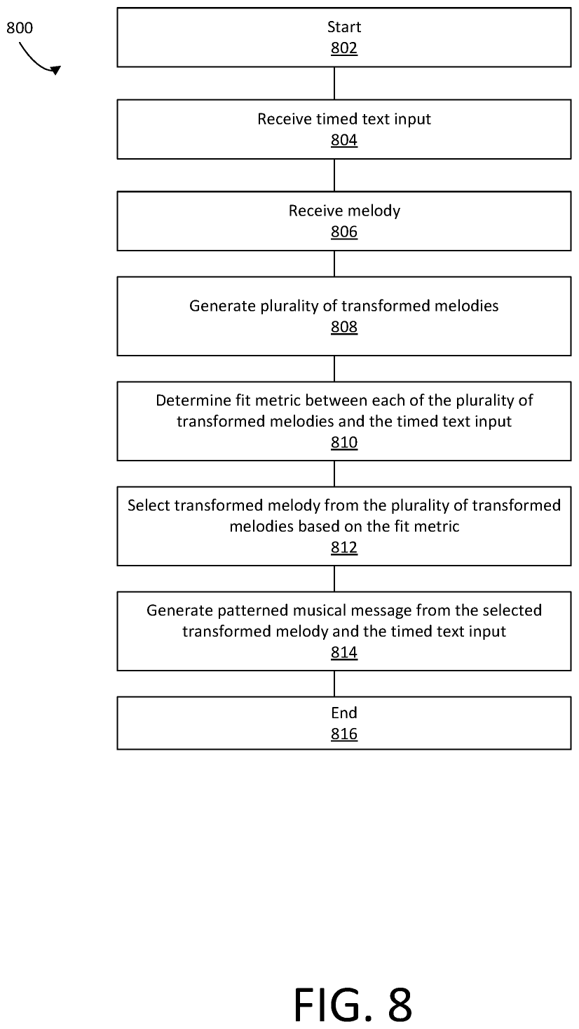

In one example, a melody segment may be transformed to generate one or more transformed melody segments. Each transformed melody segment may be analyzed to determine an optimal melody segment with which to generate a patterned musical message. Generating the one or more transformed melody segments may advantageously enable a larger group of melody segments to be analyzed to determine an optimal melody segment with which to generate the patterned musical message. An exemplary method 800 for modifying a melody in order to generate a patterned musical message is shown in FIG. 8. However, it is to be appreciated that alternate examples of modifying a melody in order to generate a patterned musical message are within the scope of this disclosure, and that the process 800 has been provided for purposes of explanation only.

At step 802, the method begins.

At step 804, a timed text input is received. The timed text input may be generated from a text input, and may be generated according to steps 204-214. For example, text input may be received by accessing a text file or other computer file such as an image or photo in which the text is stored. The information from which the timed text input is generated (for example, a text file, computer file, etc.), or a portion thereof, may be selected by a user. The text may be formatted or unformatted. The text may be received via a wired or wireless connection over a network, or may be provided on a memory disk. In other embodiments, the text may be typed or copy-and-pasted directly into a device by a user. In still other embodiments, the text may be obtained by capturing an image of text and performing optical character recognition (OCR) on the image. The text may be arranged into sentences, paragraphs, and/or larger subunits of a larger work.

The text input is converted into a phonemic representation, as can be represented by any standard format such as ARPABET, IPA or SAMPA. A plurality of spoken pause lengths and a plurality of spoken phoneme lengths are determined for the text input. The length of the pauses and the phonemes represented in the text input may be determined with the help of open source software or other sources of information regarding the prosodic, syntactic, and semantic features of the text or voice. The process may involve a lookup table that synthesizes duration information about phonemes and pauses between syllables, words, sentences, and other units from other sources which describe normal speech. The process may also involve a neural sequence-to-sequence model, for instance, a transformer or LSTM trained to predict sequences of durations and pauses from sequences of words (which may be mapped to an embedding space). In some examples, the spoken length of phonemes may be determined and/or categorized according to their position in a larger syntactic unit (e.g., a word or sentence), their part of speech, or their meaning. The plurality of spoken pause lengths is mapped to a respective plurality of sung pause lengths as discussed above. The plurality of spoken phoneme lengths is mapped to a respective plurality of sung phoneme lengths. The mapping may represent, for a spoken phoneme of a given length, a range of optimal lengths for the phoneme when sung.

As discussed above, the plurality of spoken pause lengths and the plurality of spoken phoneme lengths applied may be used to determine the value of several alternative or complementary parameters. Those parameters may include optimal breaks between sentences, optimal tempo, optimal time signature, optimal pitch range, and optimal length of phonemes, where optimality is measured with respect to facilitating comprehension and/or recollection. In some cases, a number of these factors may be applied, possibly with relative weights, in mapping the plurality of spoken pause lengths and the plurality of spoken phoneme lengths.

A timed text input is then generated from the plurality of sung pause lengths and the plurality of sung phoneme lengths. In particular, each phoneme and pause in the text input is stored in association with its respective optimal timing (i.e., length) information determined in the previous steps. The timed text input (i.e., the text input and associated timing information) may be stored in an array, a record, and/or a file in a suitable format.

In other embodiments, the input text may be provided or derived from spoken or textual input by the user. In one example, the input message may be speech captured by a microphone (e.g., microphone 110) and stored in a memory (memory 130). In some examples, the intermediate step of parsing the input message spoken by the user into components parts of speech may be performed as a precursor to or in conjunction with generating the timed text input. In other examples, parsing the spoken input into text may be modified or omitted, and the waveform of the input message itself may simply be pitch-shifted according to certain rules and/or constraints as discussed herein. In either case, it will be appreciated that a user's spoken input message may be mapped to and output as a melody in real-time or near-real-time as discussed herein.

At step 806, a melody is received. A melody may be made up of one or more melody segments. Each melody segment may be thought of as the definition of a song, melody, or portion thereof, such as a phrase or motif, and may comprise a score as discussed above. For example, the melody segment may include, for each note in the melody, a number of syllables associated with the note, a duration of the note, an absolute or relative pitch of the note, and any other timing information for the note (including any rests before or after the note).

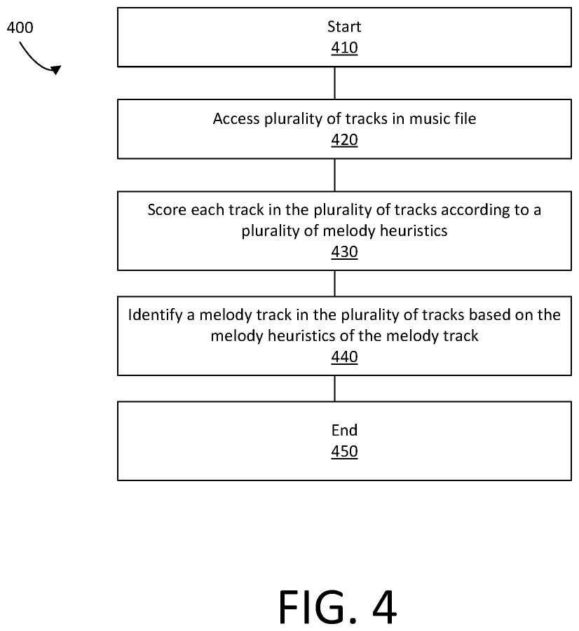

The melody segments may be accessed in a MIDI file or other format. In some examples, the plurality of melody segments may be extracted from the MIDI file according to method 400 described with respect to FIG. 4. In addition to a melody line, a musical score or other information for providing an accompaniment to the melody may be accessed. For example, a stored backing track may be accessed and prepared to be played out in synchronization with the melody segments as described in later steps. The score may include backing track information, or may provide a link to a prerecorded backing track and/or accompaniment. For example, a backing track may be provided along with the patterned musical message, such as by simulating drums, piano, backing vocals, or other aspects of the composition or its performance. In some embodiments, the backing track may be one or more short segments that can be looped for the duration of the patterned musical message. In some examples, the score is stored and presented according to a technical standard for describing event messages, such as the MIDI standard. Data in the score may specify the instructions for music, including a note's notation, pitch, velocity, vibrato, and timing/tempo information.

In other examples, a pre-existing melody may not be accessed, but rather a melody may be generated using constraint programming, logic programming, and/or generative neural models. Melodies may be generated according to rules typically found in a particular genre, and tempo, intervals, and other factors affecting comprehension and/or recall may be optimized based on a particular disorder being treated. For example, it may be determined (for example, by acquiring and implementing feedback to refine a melody-generation process over time and/or by analyzing a speed of learning over time) that a subject having a particular disorder is more likely to comprehend information when it is presented in musical form at approximately 100 beats-per-minute and utilizing a I-IV-I-IV-V-IV-I musical structure as may be found in the blues genre. It also may be determined that heuristics not typically applied in traditional musical analysis, like entropy of n-grams of intervals, may prove relevant in determining which notes should be generated. These considerations can be stored in the form of rules and used to generate a melody in real-time in order to optimize the subject's comprehension of the song and/or the visual text representation and its supporting images. The ruleset may include interval or scale information to be used in pitch shifting the audio input message. For example, the ruleset may reflect the fact that a particular genre uses a blues scale, or frequently incorporates a minor seventh (as part of a dominant seventh chord). The ruleset can then be used to pitch shift certain syllables appropriately. The ruleset may reflect that different sections or measures of a musical performance of the genre may feature different phrasings or scales. For example, the ruleset may reflect the twelve-bar blues progression, with the appropriate pitches and scale being used in each measure differing based on the measure's location within the progression. In various examples, the ruleset may learn over time based on feedback information. For example, a machine-learning and/or artificial-intelligence process may be implemented to update and refine the ruleset over time as feedback information is acquired.

At step 808, a plurality of transformed melodies is generated from the melody. It will be appreciated that the melody may be a melody segment (e.g., a phrase or motif) derived from a longer melody. The plurality of transformed melodies may be generated through musical transformation of the melody. In particular, each of the transformed melodies may represent a variation of the melody. The variation may be, for example, an augmentation, diminution, inversion, elaboration, and/or simplification of the melody or a sub-portion (e.g., phrase or notes) thereof. In some examples, the sub-portions of the melody may be re-arranged and/or re-ordered. Certain constraints may be applied in generating the plurality of transformed melodies thus enhancing song comprehension and melodic appreciation of the melody. For example, the transformed melodies may retain the prolongational structure or contour of the original melody. However, in some examples the transformed melodies may have different harmonic and rhythmic profiles and/or number of notes.

In one example, stochastic models (e.g., a Markov model) may be applied to the melody to generate the transformed melodies. In particular, a stochastic model may be used to generate a number of transformed melodies as variants of the melody. In one example, about 30 transformed melodies are generated from the melody.

At step 810, a fit metric is determined between each transformed melody of the plurality of transformed melodies and the timed text input, and at step 812, a transformed melody is selected from the plurality of transformed melodies based on the fit metric of the selected transformed melody. In some examples, the fit metric is determined using a heuristic method to perform pattern matching efficiently. For example, a geometric-hashing method may be executed with each of the plurality of transformed melodies generated in step 808 being described as a feature vector. In one example, each of the plurality of transformed melodies is described as a binary bit vector. Each position in the binary bit vector may represent a time position in the transformed melody. The binary bit vector may store a “1” at each time position in the transformed melody where a note starts, and may store a “0” in all other positions.

It will be appreciated that the binary bit vector representation of a transformed melody may be a sparse vector. Because a sparse vector may be less optimal for use in the hashing function used in determining the fit metric, neural auto encoders are used in some embodiments to first transform the sparse vectors into dense vectors. In some examples, the loss function of the neural auto encoder may be an edit distance.

A binary bit vector may also be generated from the timed text input, with a “1” stored at each time position in the timed text input where a phoneme begins, and a “0” stored at all other positions.

To generate the fit metric, the bit vector for each transformed melody may be compared to the timed text input, to determine which transformed melody represents the best fit for the timed text input. In one example, the best fit may represent a transformed melody in which the onset of notes in the transformed melody is the closest match to the onset of phonemes in the timed text input. In another example, a best fit may represent a transformed melody in which the onset of notes in the transformed melody optimally matches phonemes favoring the natural prosody of the timed text input. In other examples, a best fit may represent other similarities between transformed melodies and timed text inputs. The fit may be determined by taking the cosine similarity between the respective dense vectors, with the transformed melody represented by the bit vector corresponding to the dense vector having the highest cosine similarity to the bit vector of the timed text input being selected as the best fit. In some examples, this step 810 may be performed as part of, or in conjunction with, step 216 of generating the plurality of matching metrics in method 200.

At step 814, a patterned musical message is generated from the selected transformed melody and the timed text input. For example, each phoneme of the timed text input may be pitch shifted according the corresponding notes(s) in the melody segment. The phoneme is set to the melody using phonetic transcription codes, such as ARPABET. The patterned musical message, with or without accompaniment, may then be output as a sound file, such as a .WAV or .MP3 file suitable for output by a playback device. The patterned musical message may be encoded with timestamps indicating a relative or absolute time at which each portion (e.g., note) of the melody is to be output.

At step 816, the method ends.

The embodiments described here involve many hyper parameters, such as pace, tempo, etc. It can be understood that various valuable objectives, such as novelty, musicality, comprehensibility, serenity, etc., can be thought of as a (possibly complex) function of these hyperparameters (i.e., the tempo and scale-type used interact in a complex way to produce a certain level of comprehensibility when tuned to specific values). It will also be understood that it may not be possible to determine entirely through intuition what the optimal setting of these hyperparameters is, but it may be the case that there is data regarding the objective value function (how well the music does with respect to an objective) at specific settings of the hyperparameters. This source of data may be able to be queried at arbitrary points in the parameter space, enabling active learning. In this case, it will be possible to use one of the standard techniques for optimization of black-box functions, including Bayesian optimization. Other methods including MCMC or grid search may be used even when the choice of sampled parameters is not made in an active learning setting.

Accordingly, in various examples, text inputs may be used to generate patterned musical messages to, for example, aid in comprehension of the text inputs. In some examples, text inputs may be used to generate audible signals that affect a mental state of an individual in addition to, or in lieu of, aiding comprehension of a text. As used herein, a “mental state” refers to a current state-of-mind and mood of an individual, such as whether an individual is experiencing fear or stress.

A mental state of an individual is affected by various portions of the human brain. For example, the amygdala regulates immediate responses to fear, and is often associated with stress and psychiatric diseases. The amygdala is straddled by the hippocampus, which provides relational information integral to a “fight, flight, or freeze” response. The hippocampus is thus key to a cognitive form of innate immunity, shaped by the evolutionary pressure of social-distancing to protect against epidemics, predation, strife, and so forth, as well as signaling and communication.

A sub-part of the amygdala is the bed nucleus of the stria terminalis (BNST), also referred to as the extended amygdala, which is located in the basal forebrain and consists of 12 to 18 sub-nuclei. These sub-nuclei are rich with specific neuronal subpopulations, and are characterized by epigenetics, transcriptomics, proteins, receptors, neurotransmitters, and transporters. The BNST is integral to a range of behaviors including a fight, flight, or freeze response, extended-duration fear states, and social behavior, all of which are crucial characteristics of human psychiatric diseases. Although the BNST is crucial in playing a protective role in humans (for example, by providing a fear response to potentially dangerous triggers), the BNST may become sensitive to certain associative triggers that pose a minimal threat and thus induce unneeded and often overwhelming mental states.

It may be advantageous to adopt adaptative mental schemes to control or temper transitions into and out of certain mental states. For example, it may be advantageous to control and/or temper transitions in and out of a mental state which may not be advantageous to an individual, such as such overwhelming (and often unnecessary) mental states. If an individual typically reacts in an unhelpful manner to a particular trigger, such as by experiencing debilitating stress or fear in response to relatively benign triggering events or conditions, it may be advantageous to enable a user to react in a different, more helpful manner to the particular trigger, or to transition out of such a mental state more quickly.

There are at least two methods of controlling or tempering such transitions into and out of particular mental states. A first method includes modulating high-roads traveling out of an individual's neo-cortex to provide logical explanations to emotional triggers such that the individual may avoid obsessive and repetitive worries, which may be undesirable or disadvantageous to a user. High-road transitions, which may include transitions in an individual's mental state caused by such neo-cortex-based responses, may be controlled via talk therapy, mindfulness, cognitive behavioral therapy, reading or listening to certain texts (which may be presented as prose or poetry, for example), and so forth. High-road transitions may be relatively sparse and slow, but likely to result in durable, long-lasting transitions in an individual's mental state.

A second method includes modulating low-roads traveling out of the individual's hypothalamus to provide a bio-chemical control signal in the individual's brain to alter a mental state of the individual. Low-road transitions, which may include transitions in an individual's mental state caused by such hypothalamus-based responses, may be controlled via psychiatric drugs, breathing exercise, meditation, chanting (for example, in an unfamiliar language), and so forth. Low-road transitions may be relatively rapid as compared to high-road transitions, but may result in relatively impermanent and brief transition in an individual's mental state.

Examples of the disclosure enable a mental state of an individual to be controlled through modulation of both of an individual's high-roads and low-roads in part by outputting patterned musical messages, thereby achieving the benefits of both high-road transitions and low-road transitions. In one example, a text input is selected by a user, and the text is converted into an audible output including one or more patterned musical messages, which may be sung, chanted, or a combination of both. The audible output may be synchronized with breathing exercises and/or cycles, such as by synchronizing to periods of an individual inhaling, holding one's breath, and exhaling. A mental exercise that dictates generation of the audible output may be updated based on user feedback to optimize the mental exercise for controlling the individual's mental state by updating parameters of the mental exercise. Updating hyper-parameters of a machine-learning algorithm for optimizing the mental exercise may be performed over time as feedback indicative of an individual's mental state is acquired. For example, Bayesian optimization may be applied to update the parameters based on user feedback information.

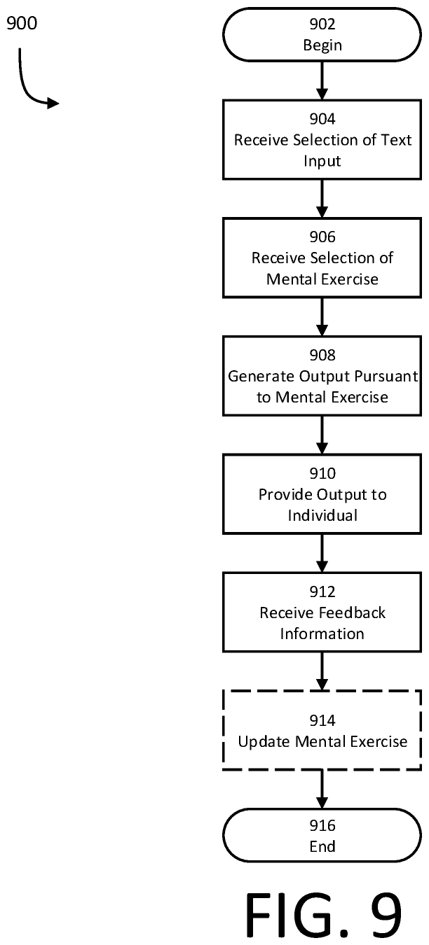

FIG. 9 illustrates a process 900 of modulating a mental state of an individual using audible outputs according to an example. In some examples, the process 900 may be executed by an electronic device such as the device 100.

At act 902, the process 900 begins.

At act 904, the device 100 receives a selection of a text input. An individual operating the device 100 may select a text. For example, the device 100 may present one or more texts to the individual via the user interface 150 and/or display 180, and the individual may select at least one of the texts via the user interface 150. Texts may be received by the device 100 in a manner substantially similar to that discussed above with respect to act 204. The texts may be stored in storage accessible to the device 100, such as in local storage (for example, the memory 130), remote storage (for example, on a device accessible via the interface 190), or a combination of both. In another example, act 904 may include a user selecting a text by inputting the text into the user interface 150, such as by typing the text into the user interface 150. In some examples, an input may be provided to the device 100 as a spoken input, or in another format other than a text input, and the input may be transcribed or otherwise transformed into a text input. In various examples, the individual may additionally or alternatively select multiple texts and/or sub-portions (or “phrases”) of a text at act 904.

At act 906, the device 100 receives a selection of a mental exercise to execute with respect to the text input. A mental exercise may represent a module of choice that may be selected by a user. The device 100 may provide several mental exercises for an individual to select. One or more of the mental exercises may correspond to a desired or current mental state of an individual. For example, one mental exercise may correspond to de-stressing an individual, and the individual may select the mental exercise if the individual is currently experiencing stress. In another example, a mental exercise may correspond to de-escalating a fear response in an individual, and the individual may select the mental exercise if the individual is currently experiencing fear. In some examples, the device 100 may automatically select a mental exercise. For example, the device 100 may receive information indicative of a state of a individual (for example, breathing-rate information, heart-rate information, blood-pressure information, and so forth) and select an appropriate mental exercise, such as a de-stressing mental exercise if the information indicative of the state of the individual indicates that the individual is experiencing stress.

In other examples, mental exercises may not be limited to desired or current mental states. Mental exercises may additionally or alternatively correspond to certain conditions of an individual. For example, a mental exercise may be designed to facilitate comprehension of the text input for individuals with dyslexia, an autism-spectrum disorder, or other conditions. For example, a mental exercise may be selected to aid in learning and/or comprehension of a selected text, which may or may not be based on a current or desired mental state or condition of the individual. In other examples, other mental exercises may be selected on other bases at act 906.

At act 908, an output is generated from the text input pursuant to the mental exercise. The output may include one or more patterned musical messages generated pursuant to the processes 200 and/or 800, for example, and/or textual and/or visual information. In some examples, multiple patterned musical messages may be generated. For example, a first patterned musical message may be executed to generate a sung output using the text input received at act 904. A second patterned musical message may be executed to generate a chanted input using the text input for a certain phrase from the text input. The patterned musical messages may be output simultaneously, sequentially, alternately, a combination thereof, or based on other schemes. As discussed above, patterned musical messages may be accompanied by textual and/or visual information. For example, the patterned musical messages may be output while a textual breathing exercise (for example, instructing an individual when to inhale and exhale) is visually displayed, either literally (for example, by displaying the words “inhale” and/or “exhale”) or symbolically (for example, by illuminating a first LED to prompt the individual to inhale, and a second LED to prompt the individual to exhale). In other examples, other types and/or combinations of patterned musical messages and textual or visual outputs are provided.

The mental exercise may dictate how the output is generated, such as by specifying output-generation parameters (including, for example, hyperparameters) used to generate an output. Output-generation parameters may indicate a number and type of outputs, and/or specify certain parameters of the outputs, such as by controlling or influencing the mapping of spoken pause or phoneme lengths to sung and/or chanted pause or phoneme lengths, controlling or influencing the matching metrics generated for each melody segment, and so forth. For example, the mental exercise may dictate that a text input is to be converted into a sung patterned musical message and a chanted patterned musical message, or a patterned musical message that is both sung and chanted. The mental exercise may dictate output-generation parameters of the patterned musical message(s), such as by dictating a melody according to which the patterned musical message is generated. For example, the mental exercise may specify a particular tempo, rhythm, harmony, counterpoint, descant, chant, spoken cadence (e.g., beat poetry), timbre, and so forth, of each of the sung and chanted patterned musical messages. In an example in which the patterned musical message(s) are output in combination with a visual or textual output, such as a visual output to guide an individual's breathing pattern, the mental exercise may specify parameters of the breathing pattern, such as a number, duration, and frequency of inhale-and-exhale cycles. Accordingly, it is to be appreciated that a mental exercise selected by a user may dictate output-generation parameters that control any aspect of how an output is generated and/or output based on a text input.

At act 910, the output generated at act 908 is provided to an individual. The output may be provided to the individual via one or more components of the device 100. For example, a visual and/or textual output may be displayed via the user interface 150, display elements 170, and/or display 180, and patterned musical message(s) may be output via the speaker/output 140.

At act 912, feedback information indicative of an effect of the output on the individual is received. The feedback information may be used to determine an efficacy of the mental exercise. For example, if the mental exercise is a de-stressing mental exercise, the feedback information may be indicative of a stress level of the individual. The feedback information may be used to determine an efficacy of the mental exercise, such as by determining whether the individual has experienced a reduction in stress after experiencing a de-stressing mental exercise. As discussed below, the feedback information may be used to optimize a mental exercise such that the efficacy of the mental exercise is maximized.

Feedback information may include information provided directly and actively by the individual. For example, the feedback information may include a subjective level of satisfaction selected by the individual for a mental exercise, such as a user-selected rating.

Feedback information may also include information indicative of inputs received from the individual, such as metadata indicative of the user inputs. For example, a mental exercise to provide a sung output indicative of a text to individuals with dyslexia may enable the individual to change aspects of the sung output, such as the genre, tempo, and so forth, of the sung output. The feedback information may include metadata indicative of the aspects changed and selected by the individual. For example, if the individual repeatedly cycled through genres before arriving at a classical-music genre, and thereafter stopped changing the genre of the sung output, the feedback information may indicate the duration of time that the individual used each genre. The feedback information may be used to determine that it may be beneficial to generate patterned musical messages pursuant to the mental exercise using the most frequently used genre in the future, in this example.

Feedback information may also include information acquired without a direct input from the individual, such as information measured from the individual. Such information may include biological information such as brain-scan information, image- or video-analysis information (for example, indicative of the individual's eye movements, facial expressions, gestures, and so forth), verbal-cue and haptic-cue information, blood-pressure information, breathing-pattern information, heart-rate information, skin-conductivity information, or other types of information that may be affected or influenced by a mental state of the individual, or may otherwise be indicative of an effect that a mental exercise is having on a mental state of the individual. The feedback information may be acquired by the device 100 directly in some examples, or may be acquired by an external device or system and provided to the device 100 via the interface 190.

At optional act 914, the mental exercise selected at act 906 is updated. The mental exercise may be updated based on the feedback information received at act 912 using a machine-learning and/or artificial-intelligence algorithm to optimize the mental exercise. The mental exercise may alternately or additionally be updated based on general information other than the feedback information, such as a body-mass index of the individual, a gender of the individual, genetic polymorphisms of the individual, a cultural background of the individual, a language-of-choice of the individual, a time-of-day at which the mental exercise is executed, and so forth.

As discussed above, the mental exercise may dictate how outputs are generated based on certain inputs. Updating the mental exercise may include modifying the manner by which the mental exercise dictates the generation of outputs. Each mental exercise may therefore be optimized for a particular user by continuously acquiring feedback information indicative of an efficacy of the mental exercise, and optionally updating the mental exercise based on the feedback information and/or general information. For example, where a machine-learning and/or artificial-learning algorithm is implemented, a computing device executing the process 900 may continuously automatically refine one or more mental exercises as feedback information is acquired and used to update the machine-learning and/or artificial-intelligence algorithm(s).

In some examples, the mental exercise may be updated in real-time based on the feedback information. For example, the mental exercise may be updated and the process 900 may return to act 908 to generate an output based on the input pursuant to the updated mental exercise such that an evolving output is continuously provided to a user, albeit according to a repeatedly updated mental exercise.

The mental exercise may be updated at least in part by updating output-generation parameters such as pace, tempo, and so forth. The output-generation parameters may further include hyperparameters of a machine-learning and/or artificial-intelligence algorithm executed at act 914 to update and/or optimize the mental exercise. As discussed above, various valuable objectives, such as novelty, musicality, comprehensibility, serenity, and so forth, can be thought of as a (possibly complex) function of these output-generation parameters (that is, the tempo and scale-type used interact in a complex way to produce a certain level of comprehensibility when tuned to specific values). It will also be understood that it may not be possible to determine entirely through intuition what the optimal setting of these output-generation parameters is, but it may be the case that there is data regarding the objective value function (how well the music does with respect to an objective) at specific settings of the output-generation parameters. This source of data may be able to be queried at arbitrary points in the parameter space, enabling active learning.

In this case, it will be possible to use function-optimization techniques such as Bayesian optimization. Bayesian optimization is a sequential-design strategy for global optimization of black-box functions. Bayesian optimization may be applied to update output-generation parameters of the process according to which the mental exercise dictates the generation of outputs based on inputs. For example, Bayesian optimization may be applied to alter a melody according to which an output is sung and/or chanted, or may be applied to alter breathing instructions provided to an individual, or may be applied to any other aspect of the output provided to the individual. Other methods including MCMC or grid search may be used even when the choice of sampled parameters is not made in an active learning setting.

In some examples, optional act 914 is not executed, and the process 900 instead continues from act 912 to act 916. For example, the feedback information may indicate that the individual is responding as desired to a mental exercise, and that no additional updates are desirable or necessary.

At act 916, the process 900 ends.

Accordingly, a mental exercise may be executed with respect to a text input selected by a user. The mental exercise may dictate output-generation parameters according to which one or more patterned musical messages are generated using the text input. For example, the output-generation parameters may dictate properties of the one or more patterned musical messages such as pace, tempo, genre, melody, and so forth. These output-generation parameters may be correlated to a particular desired result of a mental exercise, such as by being correlated to reducing stress, minimizing anxiety, aiding in learning or comprehension, and so forth. Feedback information indicative of an efficacy of the mental exercise may be acquired and used in connection with a statistical-optimization technique to update output-generation parameters of the mental exercise. The mental exercise may thus be repeatedly updated based on user feedback information such that a mental exercise is optimized for a particular user according to the user's response to the mental exercise.

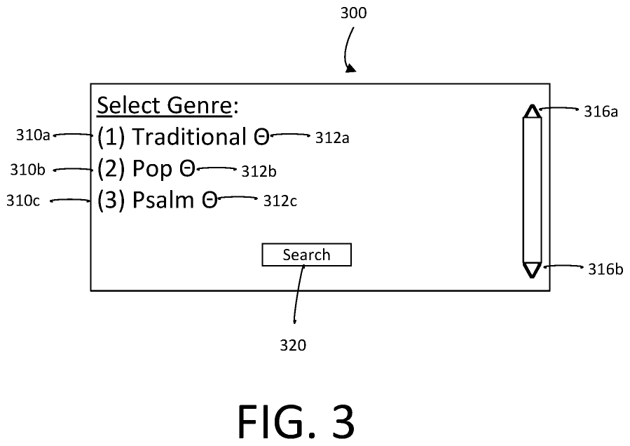

An exemplary user interface 300 for selecting a particular genre is shown in FIG. 3. The user interface 300 includes a list of selectable genres 310a-c, which may be selected by touching or otherwise interacting with the user interface. Additional information about the genre may be displayed by clicking on the corresponding information indicator 312a-c next to each genre. Controls 316a,b allow the user to scroll up and down or otherwise navigate the list, and a search functionality may be provided by interacting with control element 320. The search functionality may allow the user to search for available genres.

It will be appreciated that a broad selection of melodies and melody segments will facilitate optimal matching of the time text input to melody segments (e.g., in steps 214 and 216 discussed above), and that such a broader selection also increases user engagement and enjoyment. It will also be appreciated that identifying melodies for inclusion in the pool of available options may be time-intensive, since a desired melody may be provided in available music alongside rhythm and other tracks. For example, a Musical Instrument Digital Interface (MIDI) music file for a particular song may contain a melody track along with other instrumentation (e.g., a simulated drum beat or bass line), and one or more harmony lines. There is therefore an advantage to providing an automatic method of identifying a melody among a collection of tracks forming a musical piece, in order to add additional melody segments to the collection available for matching to the timed text input as discussed above. This is accomplished by detecting one or more characteristics of a melody within a given musical line and scoring the musical line according to its likelihood of being a melody.

A method 400 of determining a melody track in a music file is described with reference to FIG. 4.

At step 410, the method begins.

At step 420, a plurality of tracks in a music file are accessed. For example, a MIDI file, a musicXML file, abc format file or other file format, may be accessed and all of the individual lines as defined by the channels/tracks in the MIDI file will be stored and accessed. Each of these lines can be evaluated as a possible melody line.

At step 430, each of the plurality of tracks is scored according to a plurality of melody heuristics. The plurality of melody heuristics may represent typical identifying characteristics of a melody. For example, the melody heuristics may represent the amount of “motion” in the melody, the number of notes, the rhythmic density (both in a given section and throughout the piece), the entropy (both in a given section and throughout the piece), and the pitch/height ambitus of the track. The melody heuristics may score a track according to a number of specific criteria that quantify those characteristics. For example, a track may be scored according to the number of interval leaps greater than a certain amount (e.g., 7 semitones); a track with a greater number of such large jumps may be less likely to be the melody. In another example, the track may be scored according to its total number of notes; a track having more notes may be more likely to be the melody. In another example, the track may be scored according to a median number of notes with no significant rest in between them; a track with fewer rests between notes may be more likely to be the melody. In another example, the track may be scored according to a median Shannon entropy of every window of the melody between 8 and 16 notes long; a track with a higher entropy may be more likely to be the melody. In another example, the track may be scored according to a number of notes outside of a typical human singing range (e.g., notes outside of the range of MIDI pitches from 48 to 84); a track with more unsingable notes may be less likely to be the melody. Other measurements that could be used include mean, median, and standard deviation of length of note durations, note pitches, and absolute values of intervals between notes, or other mathematical operators on the contents of the MIDI file.

A subscore may be determined for each of these and other criteria, and aggregated (e.g., summed) to a melody heuristic score for the track.

At step 440, a melody track is identified from among the plurality of tracks based at least in part on the plurality of melody heuristics for the melody track. For example, after each candidate track has been scored, the track with the highest melody heuristic score may be identified as the melody track. In some examples, where more than one track has a sufficiently high melody heuristic score, the candidate melody tracks may be presented to a user graphically, or may be performed audibly, so that the user can select the desired/appropriate melody track.

At step 450, the method ends.

After the melody track is identified, it may be split into melody segments, stored, and used to match with portions of timed text inputs as discussed above with reference to FIGS. 2A and 2B.

### Exemplary Implementations in a Computer Accessible Medium

Processes described above are merely illustrative embodiments of systems that may be used to execute methods for transposing spoken or textual input to music. Such illustrative embodiments are not intended to limit the scope of the present invention, as any of numerous other implementations exist for performing the invention. None of the embodiments and claims set forth herein are intended to be limited to any particular implementation of transposing spoken or textual input to music, unless such claim includes a limitation explicitly reciting a particular implementation.

Processes and methods associated with various embodiments, acts thereof and various embodiments and variations of these methods and acts, individually or in combination, may be defined by computer-readable signals tangibly embodied on a computer-readable medium, for example, a non-volatile recording medium, an integrated circuit memory element, or a combination thereof. According to one embodiment, the computer-readable medium may be non-transitory in that the computer-executable instructions may be stored permanently or semi-permanently on the medium. Such signals may define instructions, for example, as part of one or more programs, that, as a result of being executed by a computer, instruct the computer to perform one or more of the methods or acts described herein, and/or various embodiments, variations and combinations thereof. Such instructions may be written in any of a plurality of programming languages, for example, Java, Python, Javascript, Visual Basic, C, C #, or C++, etc., or any of a variety of combinations thereof. The computer-readable medium on which such instructions are stored may reside on one or more of the components of a general-purpose computer described above, and may be distributed across one or more of such components.

The computer-readable medium may be transportable such that the instructions stored thereon can be loaded onto any computer system resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a processor to implement the above-discussed aspects of the present invention.

The computer system may include specially-programmed, special-purpose hardware, for example, an application-specific integrated circuit (ASIC) or a field programmable gate array (FPGA). Aspects of the invention may be implemented in software, hardware or firmware, or any combination thereof. Further, such methods, acts, systems, system elements and components thereof may be implemented as part of the computer system described above or as an independent component.

A computer system may be a general-purpose computer system that is programmable using a high-level computer programming language. A computer system may be also implemented using specially programmed, special purpose hardware. In a computer system there may be a processor that is typically a commercially available processor such as the Pentium class processor available from the Intel Corporation. Many other processors are available. Such a processor usually executes an operating system which may be, for example, any version of the Windows, iOS, Mac OS, or Android OS operating systems, or UNIX/LINUX available from various sources. Many other operating systems may be used. The RETM implementation may also rely on a commercially available embedded device, such as an Arduino or Raspberry Pi device.

Some aspects of the invention may be implemented as distributed application components that may be executed on a number of different types of systems coupled over a computer network. Some components may be located and executed on mobile devices, servers, tablets, or other system types. Other components of a distributed system may also be used, such as databases or other component types.

The processor and operating system together define a computer platform for which application programs in high-level programming languages are written. It should be understood that the invention is not limited to a particular computer system platform, processor, operating system, computational set of algorithms, code, or network. Further, it should be appreciated that multiple computer platform types may be used in a distributed computer system that implement various aspects of the present invention. Also, it should be apparent to those skilled in the art that the present invention is not limited to a specific programming language, computational set of algorithms, code or computer system. Further, it should be appreciated that other appropriate programming languages and other appropriate computer systems could also be used.

One or more portions of the computer system may be distributed across one or more computer systems coupled to a communications network. These computer systems also may be general-purpose computer systems. For example, various aspects of the invention may be distributed among one or more computer systems configured to provide a service (e.g., servers) to one or more client computers, or to perform an overall task as part of a distributed system. For example, various aspects of the invention may be performed on a client-server system that includes components distributed among one or more server systems that perform various functions according to various embodiments of the invention. These components may be executable, intermediate (e.g., IL) or interpreted (e.g., Java) code which communicate over a communication network (e.g., the Internet) using a communication protocol (e.g., TCP/IP). Certain aspects of the present invention may also be implemented on a cloud-based computer system (e.g., the EC2 cloud-based computing platform provided by Amazon.com), a distributed computer network including clients and servers, or any combination of systems.

It should be appreciated that the invention is not limited to executing on any particular system or group of systems. Also, it should be appreciated that the invention is not limited to any particular distributed architecture, network, or communication protocol.

Further, on each of the one or more computer systems that include one or more components of device 100, each of the components may reside in one or more locations on the system. For example, different portions of the components of device 100 may reside in different areas of memory (e.g., RAM, ROM, disk, etc.) on one or more computer systems. Each of such one or more computer systems may include, among other components, a plurality of known components such as one or more processors, a memory system, a disk storage system, one or more network interfaces, and one or more busses or other internal communication links interconnecting the various components.

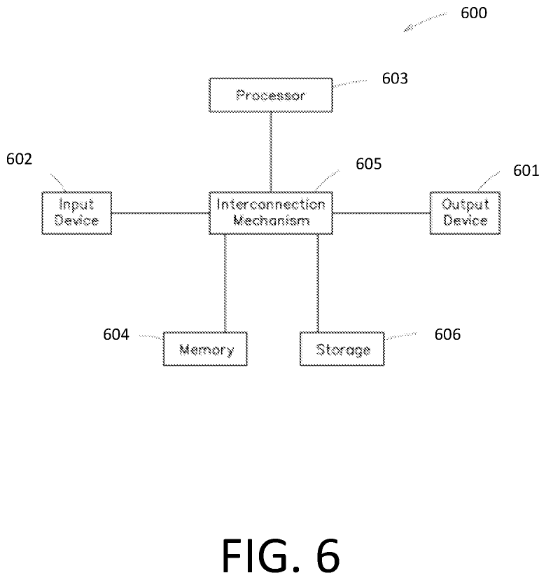

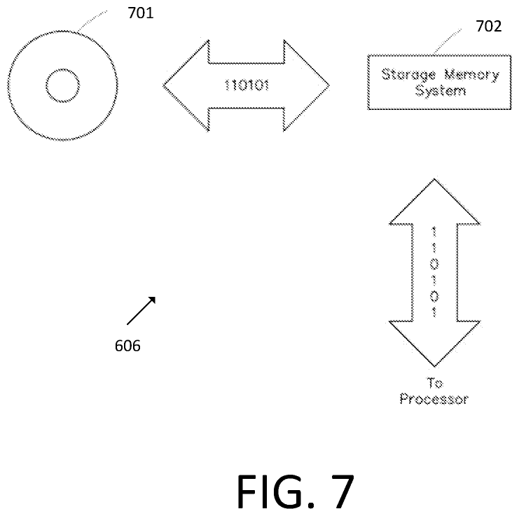

A RETM may be implemented on a computer system described below in relation to FIGS. 6 and 7. In particular, FIG. 6 shows an example computer system 600 used to implement various aspects. FIG. 7 shows an example storage system that may be used.

System 600 is merely an illustrative embodiment of a computer system suitable for implementing various aspects of the invention. Such an illustrative embodiment is not intended to limit the scope of the invention, as any of numerous other implementations of the system, for example, are possible and are intended to fall within the scope of the invention. For example, a virtual computing platform may be used. None of the claims set forth below are intended to be limited to any particular implementation of the system unless such claim includes a limitation explicitly reciting a particular implementation.

Various embodiments according to the invention may be implemented on one or more computer systems. These computer systems may be, for example, general-purpose computers such as those based on Intel PENTIUM-type processor, Motorola PowerPC, Sun UltraSPARC, Hewlett-Packard PA-RISC processors, or any other type of processor. It should be appreciated that one or more of any type computer system may be used to partially or fully automate integration of the recited devices and systems with the other systems and services according to various embodiments of the invention. Further, the software design system may be located on a single computer or may be distributed among a plurality of computers attached by a communications network.

For example, various aspects of the invention may be implemented as specialized software executing in a general-purpose computer system 600 such as that shown in FIG. 6. The computer system 600 may include a processor 603 connected to one or more memory devices 604, such as a disk drive, memory, or other device for storing data. Memory 604 is typically used for storing programs and data during operation of the computer system 600. Components of computer system 600 may be coupled by an interconnection mechanism 605, which may include one or more busses (e.g., between components that are integrated within a same machine) and/or a network (e.g., between components that reside on separate discrete machines). The interconnection mechanism 605 enables communications (e.g., data, instructions) to be exchanged between system components of system 600. Computer system 600 also includes one or more input devices 602, for example, a keyboard, mouse, trackball, microphone, touch screen, and one or more output devices 601, for example, a printing device, display screen, and/or speaker. In addition, computer system 600 may contain one or more interfaces (not shown) that connect computer system 600 to a communication network (in addition or as an alternative to the interconnection mechanism 605).

The storage system 606, shown in greater detail in FIG. 7, typically includes a computer readable and writeable nonvolatile recording medium 701 in which signals are stored that define a program to be executed by the processor or information stored on or in the medium 701 to be processed by the program. The medium may, for example, be a disk or flash memory. Typically, in operation, the processor causes data to be read from the nonvolatile recording medium 701 into another memory 702 that allows for faster access to the information by the processor than does the medium 701. This memory 702 is typically a volatile, random access memory such as a dynamic random-access memory (DRAM) or static memory (SRAM).

Data may be located in storage system 606, as shown, or in memory system 604. The processor 603 generally manipulates the data within the integrated circuit memory 604, 602 and then copies the data to the medium 701 after processing is completed. A variety of mechanisms are known for managing data movement between the medium 701 and the integrated circuit memory element 604, 702, and the invention is not limited thereto. The invention is not limited to a particular memory system 604 or storage system 606.

Although computer system 600 is shown by way of example as one type of computer system upon which various aspects of the invention may be practiced, it should be appreciated that aspects of the invention are not limited to being implemented on the computer system as shown in FIG. 6. Various aspects of the invention may be practiced on one or more computers having a different architecture or components than that shown in FIG. 6.

Computer system 600 may be a general-purpose computer system that is programmable using a high-level computer programming language. Computer system 600 may be also implemented using specially programmed, special purpose hardware. In computer system 600, processor 603 is typically a commercially available processor such as the Pentium, Core, Core Vpro, Xeon, or Itanium class processors available from the Intel Corporation. Many other processors are available. Such a processor usually executes an operating system which may be, for example, operating systems provided by Microsoft Corporation or Apple Corporation, including versions for PCs as well as mobile devices, iOS, Android OS operating systems, or UNIX available from various sources. Many other operating systems may be used.

Various embodiments of the present invention may be programmed using an object-oriented programming language, such as SmallTalk, Python, Java, C++, Ada, or C# (C-Sharp). Other object-oriented programming languages may also be used. Alternatively, functional, scripting, and/or logical programming languages may be used. Various aspects of the invention may be implemented in a non-programmed environment (e.g., documents created in HTML, XML or other format that, when viewed in a window of a browser program, render aspects of a graphical-user interface (GUI) or perform other functions). Various aspects of the invention may be implemented using various Internet technologies such as, for example, the Common Gateway Interface (CGI) script, PHP Hyper-text Preprocessor (PHP), Active Server Pages (ASP), HyperText Markup Language (HTML), Extensible Markup Language (XML), Java, JavaScript and open source libraries for extending Javascript, Asynchronous JavaScript and XML (AJAX), Flash, and other programming methods. Further, various aspects of the present invention may be implemented in a cloud-based computing platform, such as the EC2 platform available commercially from Amazon.com (Seattle, WA), among others. Various aspects of the invention may be implemented as programmed or non-programmed elements, or any combination thereof.

### Methods of Use

Described herein are real-time musical translation devices (RETMs) and related software suitable for receiving real-time input (e.g., a text, audio or spoken message) containing information to be conveyed, and converting that input to a patterned musical message (e.g., a song or melody) that may be useful to treat an indication in a user, such as a cognitive impairment, a behavioral impairment, or a learning impairment, or to generally facilitate learning.

While the embodiments discussed herein relate to translating words or text to song in order to facilitate word or syntax comprehension or memory, other methods of use should be understood to be within the scope of this disclosure. For example, in many current video games, including RPGs (role-playing games), action games, simulation games, and strategy games, users are presented with dialog with other characters in the game, with a narrator, or as a set of instructions on how to play the game. In one embodiment, the RETM may be used by game developers to convert whatever text is presented in the game to song during the course of gameplay, and for instructions and aspects of setting up and running the game. Such an embodiment may provide enhanced enjoyment of the game for both users with and without disorders. In addition, it may increase accessibility of these videogames to users with language- or text-related impairments as described above.

In another example, it will be appreciated that virtual digital assistants (e.g., Alexa by Amazon) are often interacted with, in homes and businesses, through devices such as smart speakers. Such virtual assistants may be modified according to aspects described herein to respond through song to the respondent, rather than through spoken voice, to allow optimal comprehension of the system's response, thereby returning information on products music, news, weather, sports, home system functioning and more to a person in need of song for optimal comprehension and functioning.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### ENUMERATED EMBODIMENTS

1. A method of transforming textual input to a musical score comprising:
receiving a timed text input;
receiving a melody;
generating a plurality of transformed melodies from the melody;
determining, for each of the plurality of transformed melodies, a fit metric between the respective transformed melody and the timed text input;
selecting the transformed melody from the plurality of transformed melodies based on the fit metric of the selected transformed melody; and
generating a patterned musical message from the selected transformed melody and the timed text input.

2. The method of embodiment 1, wherein generating the plurality of transformed melodies from the melody comprises:
splitting the melody into a plurality of melody subsequences (e.g., determined using heuristics for segmentation); and
generating the plurality of transformed melodies from the plurality of melody subsequences.

3. The method of any one of embodiments 1-2, further comprising:
splitting the timed text input into a plurality of timed text subsequences; and
determining, for each of the timed text sequences, a fit metric between the respective timed text sequence and each of the plurality of transformed melodies.

4. The method of embodiment 3, wherein splitting the timed text input into the plurality of timed text subsequences is iteratively performed using dynamic programming.

5. The method of any one of embodiments 1-4, wherein generating the plurality of transformed melodies from the melody is performed using a stochastic model.

6. The method of embodiment 5, wherein the stochastic model is a Markov model.

7. The method of any one of embodiments 1-6, wherein determining, for each of the plurality of transformed melodies, the fit metric for the respective transformed melody and the timed text input comprises:
determining an onset pattern of the transformed melody;
determining a set of optimal syllable lengths of the timed text input;
mapping the onset pattern of the transformed melody to a first real-valued vector;
mapping the optimal syllable lengths of the timed text input to a second real-valued vector; and
determining a cosine similarity between the first real-valued vector and the second real-valued vector.

8. The method of embodiment 7, wherein the first real-valued vector and the second real-valued vector are derived from sparse vectors, in which the only non-zero entries are at the onsets of the melody or timed text input.

9. The method of embodiment 8, wherein dense vectors are derived from the sparse vectors using a neural autoencoder, and wherein a loss function of the neural autoencoder is an edit distance loss function.

10. The method of any one of embodiments 1-9, wherein the timed text input is received as text input by a user of a device.

11. The method of any one of embodiments 1-10, wherein the timed text input is derived from speech spoken by a user and received at a microphone of a device.

12. The method of embodiment 11, where the timed text input is derived from text that is derived from speech spoken by a user and received at a microphone of a device.

13. The method of any one of embodiments 1-12, wherein generating the plurality of transformed melodies from the melody comprises augmenting, diminishing, inverting, elaborating, transposing, or simplifying at least one portion of the melody.

14. The method of any one of embodiments 1-13, wherein receiving the melody comprises automatically composing a plurality of melody subsequences using at least one of constraint programming, logic programming, or generative neural models.

15. A system for transforming textual input to a musical score comprising:
a processor; and
a memory configured to store instructions that when executed by the processor cause the processor to:
   receive a timed text input;
   receive a melody;
   generate a plurality of transformed melodies from the melody;
   determine, for each of the plurality of transformed melodies, a fit metric between the respective transformed melody and the timed text input;
   select the transformed melody from the plurality of transformed melodies based on the fit metric of the selected transformed melody; and
   generate a patterned musical message from the selected transformed melody and the timed text input.

16. A non-transitory computer-readable medium storing sequences of instruction for transforming textual input to a musical score, the sequences of instruction including computer executable instructions that instruct at least one processor to:
receive a timed text input;
receive a melody;
generate a plurality of transformed melodies from the melody;
determine, for each of the plurality of transformed melodies, a fit metric between the respective transformed melody and the timed text input;
select the transformed melody from the plurality of transformed melodies based on the fit metric of the selected transformed melody; and
generate a patterned musical message from the selected transformed melody and the timed text input.

17. A method of modulating a state of a user, the method comprising:
receiving, via a user interface, a first selection of a text;
selecting, based on at least one parameter of the user, a mental exercise;
converting, based on the selected mental exercise, the first selection of the text to at least one of a first sung sequence or a first chanted sequence; and
outputting the at least one of the sung sequence or the chanted sequence via a transducer.

18. The method of embodiment 17, wherein the at least one parameter of the user includes one of a gender of the user, a body-mass index of the user, a time of day, a genetic polymorphism of the user, a cultural background of the user, or a language chosen by the user.

19. The method of embodiment 17, further comprising:
receiving feedback information indicative of the state of the user;
modifying, based on the at least one parameter of the user and the feedback information, the mental exercise to optimize the mental exercise for modulation of the state of the user;
receiving, via the user interface, a second selection of a text;
converting, based on the modified mental exercise, the second selection of the text to at least one of a second sung sequence or a second chanted sequence; and
outputting the at least one of the second sung sequence or the second chanted sequence via the transducer.

20. The method of embodiment 19, wherein the feedback information is biological or physiological feedback information.

21. The method of embodiment 20, wherein modifying the mental exercise includes executing a Bayesian-optimization technique based on the biological or physiological feedback information.

22. The method of embodiment 21, wherein executing the Bayesian-optimization technique includes optimizing the mental exercise to modulate the state of the user.

23. The method of embodiment 22, wherein the state of the user includes a mental mood of the user.

24. The method of embodiment 23, wherein modulating the mental mood of the user includes modulating a predicted altered state of at least one region of the brain involved in managing stress, involved in verbalization information indicative of the state of the user, or involved in depression of the user including the bed nucleus of the stria terminalis (BNST) of the user.

25. The method of embodiment 24, wherein outputting the at least one of the second sung sequence or the second chanted sequence via the transducer is predicted to modulate the state of the BNST of the user.

26. The method of embodiment 22, wherein the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers.

27. The method of embodiment 19, wherein the feedback includes user-input information.

28. The method of embodiment 27, wherein the user-input information includes a user selection of a metric indicative of a satisfaction of the user with the mental exercise.

29. The method of embodiment 27, wherein the user-input information includes metadata indicative of one or more inputs provided by the user.

30. The method of embodiment 29, wherein the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.

31. A non-transitory computer-readable medium storing thereon sequences of computer-executable instructions for modulating a state of a user, the sequences of computer-executable instructions including instructions that instruct at least one processor to:
receive, via a user interface, a first selection of a text;
select, based on at least one parameter of the user, a mental exercise;
convert, based on the selected mental exercise, the first selection of the text to at least one of a first sung sequence or a first chanted sequence; and
output the at least one of the sung sequence or the chanted sequence via a transducer.

32. The non-transitory computer-readable medium of embodiment 31, wherein the at least one parameter of the user includes one of a gender of the user, a body-mass index of the user, a time of day, a genetic polymorphism of the user, a cultural background of the user, or a language chosen by the user.

33. The non-transitory computer-readable medium of embodiment 31, wherein the instructions further instruct the at least one processor to:
receive feedback information indicative of the state of the user;
modify, based on the at least one parameter of the user and the feedback information, the mental exercise to optimize the mental exercise for modulation of the state of the user;
receive, via the user interface, a second selection of a text;
convert, based on the modified mental exercise, the second selection of the text to at least one of a second sung sequence or a second chanted sequence; and
output the at least one of the second sung sequence or the second chanted sequence via the transducer.

34. The non-transitory computer-readable medium of embodiment 33, wherein the feedback information is biological or physiological feedback information.

35. The non-transitory computer-readable medium of embodiment 34, wherein modifying the mental exercise includes executing a Bayesian-optimization technique based on the biological or physiological feedback information.

36. The non-transitory computer-readable medium of embodiment 35, wherein executing the Bayesian-optimization technique includes optimizing the mental exercise to modulate the state of the user.

37. The non-transitory computer-readable medium of embodiment 36, wherein the state of the user includes a mental mood of the user.

38. The non-transitory computer-readable medium of embodiment 37, wherein modulating the mental mood of the user includes modulating a predicted altered state of at least one region of the brain involved in managing stress, involved in verbalization information indicative of the state of the user, or involved in anxiety or depression of the user including the bed nucleus of the stria terminalis (BNST) of the user.

39. The non-transitory computer-readable medium of embodiment 38, wherein outputting the at least one of the second sung sequence or the second chanted sequence via the transducer is predicted to modulate the state of the BNST of the user.

40. The non-transitory computer-readable medium of embodiment 36, wherein the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers.

41. The non-transitory computer-readable medium of embodiment 33, wherein the feedback includes user-input information.

42. The non-transitory computer-readable medium of embodiment 41, wherein the user-input information includes a user selection of a metric indicative of a satisfaction of the user with the mental exercise.

43. The non-transitory computer-readable medium of embodiment 41, wherein the user-input information includes metadata indicative of one or more inputs provided by the user.

44. The non-transitory computer-readable medium of embodiment 43, wherein the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.

What is claimed is:

1. A method of modulating a state of a user, the method comprising:
receiving, via a user interface, a first selection of a text;
selecting, based on at least one parameter of the user, a mental exercise;
converting, based on the selected mental exercise, the first selection of the text to at least one of a first sung sequence or a first chanted sequence; and
outputting the at least one of the sung sequence or the chanted sequence via a transducer.

2. The method of claim 1, wherein the at least one parameter of the user includes one of a gender of the user, a body-mass index of the user, a time of day, a genetic polymorphism of the user, a cultural background of the user, or a language chosen by the user.

3. The method of claim 1, further comprising:
receiving feedback information indicative of the state of the user;
modifying, based on the at least one parameter of the user and the feedback information, the mental exercise to optimize the mental exercise for modulation of the state of the user;
receiving, via the user interface, a second selection of a text;
converting, based on the modified mental exercise, the second selection of the text to at least one of a second sung sequence or a second chanted sequence; and
outputting the at least one of the second sung sequence or the second chanted sequence via the transducer.

4. The method of claim 3, wherein the feedback information is biological or physiological feedback information.

5. The method of claim 4, wherein modifying the mental exercise includes executing a Bayesian-optimization technique based on the biological or physiological feedback information.

6. The method of claim 5, wherein executing the Bayesian-optimization technique includes optimizing the mental exercise to modulate the state of the user.

7. The method of claim 6, wherein the state of the user includes a mental mood of the user.

8. The method of claim 7, wherein modulating the mental mood of the user includes modulating a predicted altered state of at least one region of the brain involved in managing stress, involved in verbalization information indicative of the state of the user, or involved in depression of the user including the bed nucleus of the stria terminalis (BNST) of the user.

9. The method of claim 8, wherein outputting the at least one of the second sung sequence or the second chanted sequence via the transducer is predicted to modulate the state of the BNST of the user.

10. The method of claim 6, wherein the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers.

11. The method of claim 3, wherein the feedback includes user-input information.

12. The method of claim 11, wherein the user-input information includes a user selection of a metric indicative of a satisfaction of the user with the mental exercise.

13. The method of claim 11, wherein the user-input information includes metadata indicative of one or more inputs provided by the user.

14. The method of claim 13, wherein the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.

15. A non-transitory computer-readable medium storing thereon sequences of computer-executable instructions for modulating a state of a user, the sequences of computer-executable instructions including instructions that instruct at least one processor to:
receive, via a user interface, a first selection of a text;
select, based on at least one parameter of the user, a mental exercise;
convert, based on the selected mental exercise, the first selection of the text to at least one of a first sung sequence or a first chanted sequence; and
output the at least one of the sung sequence or the chanted sequence via a transducer.

16. The non-transitory computer-readable medium of claim 15, wherein the at least one parameter of the user includes one of a gender of the user, a body-mass index of the user, a time of day, a genetic polymorphism of the user, a cultural background of the user, or a language chosen by the user.

17. The non-transitory computer-readable medium of claim 15, wherein the instructions further instruct the at least one processor to:
receive feedback information indicative of the state of the user;
modify, based on the at least one parameter of the user and the feedback information, the mental exercise to optimize the mental exercise for modulation of the state of the user;
receive, via the user interface, a second selection of a text;
convert, based on the modified mental exercise, the second selection of the text to at least one of a second sung sequence or a second chanted sequence; and
output the at least one of the second sung sequence or the second chanted sequence via the transducer.

18. The non-transitory computer-readable medium of claim 17, wherein the feedback information is biological or physiological feedback information.

19. The non-transitory computer-readable medium of claim 18, wherein modifying the mental exercise includes executing a Bayesian-optimization technique based on the biological or physiological feedback information.

20. The non-transitory computer-readable medium of claim 19, wherein executing the Bayesian-optimization technique includes optimizing the mental exercise to modulate the state of the user.

21. The non-transitory computer-readable medium of claim 20, wherein the state of the user includes a mental mood of the user.

22. The non-transitory computer-readable medium of claim 21, wherein modulating the mental mood of the user includes modulating a predicted altered state of at least one region of the brain involved in managing stress, involved in verbalization information indicative of the state of the user, or involved in depression of the user including the bed nucleus of the stria terminalis (BNST) of the user.

23. The non-transitory computer-readable medium of claim 22, wherein outputting the at least one of the second sung sequence or the second chanted sequence via the transducer is predicted to modulate the state of the BNST of the user.

24. The non-transitory computer-readable medium of claim 20, wherein the biological feedback information includes at least one of brain-scan information, image-analysis information, verbal-cue information, haptic-cue information, breathing-rate information, heart-rate information, blood-pressure information, eye-movement information, muscle-tone information, or pharmacodynamic markers.

25. The non-transitory computer-readable medium of claim 17, wherein the feedback includes user-input information.

26. The non-transitory computer-readable medium of claim 25, wherein the user-input information includes a user selection of a metric indicative of a satisfaction of the user with the mental exercise.

27. The non-transitory computer-readable medium of claim 25, wherein the user-input information includes metadata indicative of one or more inputs provided by the user.

28. The non-transitory computer-readable medium of claim 27, wherein the one or more inputs provided by the user include a selection of one or more properties of at least one of the sung sequence and the chanted sequence, the one or more properties including at least one of a pace, a tempo, a genre, a pitch, a timbre, and a duration of the at least one of the sung sequence and the chanted sequence.
